(19) United States
(12) Patent Application Publication
(10) Pub. No.: US 2024/0055095 A1
(43) Pub. Date: Feb. 15, 2024

(54) INFORMATION PROCESSING APPARATUS, SUPPORT METHOD, AND NON-TRANSITORY COMPUTER READABLE MEDIUM STORING PROGRAM

(71) Applicant: Yuki KOSAKA, (US)

(72) Inventor: Yuki KOSAKA, (US)

(21) Appl. No.: 17/766,656

(22) PCT Filed: Sep. 23, 2020
(86) PCT No.: PCT/JP2020/035736

§ 371 Date: Apr. 5, 2022

(51) Int. Cl. G16H 20/30; G16H 20/60

(52) U.S. Cl. CPC G16H 20/30; G16H 20/60

(57) ABSTRACT

An information processing apparatus capable of effectively supporting the creation of a plan for activities aimed at improving abilities is provided. A storage unit (2) stores therein past patient data in which a plurality of past patients are associated with activity targets. A long-term target determination unit (12) determines, for each of the plurality of past patients, whether or not the activity target for that past patient is similar to a long-term target for an ability improving activity performed by a target patient. A short-term target extraction unit (14) extracts a short-term target set for a past patient corresponding to the activity target determined to be similar to the long-term target for the target patient. A short-term target presentation unit (16) performs a process for presenting information about the extracted short-term target to the user.

### TECHNICAL FIELD

The present invention relates to an information processing apparatus, a support method, and a non-transitory computer readable medium storing a program.

### BACKGROUND ART

In a facility in which a patient or the like performs activities aimed at improving his/her abilities including physical functions, such as rehabilitation (e.g., a rehabilitation training or a rehabilitation therapy) (hereinafter also referred to as “rehab”) (e.g., in a rehabilitation hospital), therapists such as physical therapists sometimes create rehabilitation plans. In such a case, a person who has created a rehabilitation plan needs to examine the contents thereof based on his/her experience or advice from other therapists. Therefore, it takes time to examine the rehabilitation plan.

In regard to this, Patent Literature 1 discloses a medical service support system. The medical service support system disclosed in Patent Literature 1 includes a terminal device and a server. The server includes a long-term target acquisition unit that acquires a long-term target for a medical service from the terminal device, a short-term target candidate acquisition unit that acquires short-term target candidates for achieving the long-term target, and a short-term target determination unit that determines a short-term target candidate which the patient or the like needs to achieve first as a first short-term target.

### CITATION LIST

### Patent Literature

Patent Literature 1: Japanese Unexamined Patent Application Publication No. 2018-190102

### SUMMARY OF INVENTION

### Technical Problem

In the technology disclosed in Patent Literature 1, short-term target candidate information for achieving long-term target information is acquired from a long-term target pattern table. Therefore, to use the technology disclosed in Patent Literature 1, it is necessary to create the long-term target pattern table in advance. Note that the long-term target pattern table in Patent Literature 1 is a correspondence table in which long-term targets are associated with short-term target candidates. It requires experience for a therapist or the like to create such a correspondence table as it is very complicated. Therefore, it may be impossible to effectively support the creation of a plan for activities aimed at improving abilities by using the technology disclosed in Patent Literature 1.

The present disclosure has been made to solve the above-described problem, and an object thereof is to provide an information processing apparatus, a support method, and a program capable of effectively supporting the creation of a plan for activities aimed at improving abilities.

### Solution to Problem

An information processing apparatus according to the present disclosure includes: storage means for storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients: long-term target determination means for determining whether or not the activity target of each of the plurality of past patient is similar to a long-term target of the activity of a target patient: short-term target extraction means for extracting the short-term target set for the past patient corresponding to the activity target determined to be similar to the long-term target for the target patient: and short-term target presentation means for performing a process for presenting information about the extracted short-term target.

Further, a support method according to the present disclosure includes: storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients: determining whether or not the activity target of each of the plurality of past patient is similar to a long-term target of the activity of a target patient: extracting the short-term target set for the past patient corresponding to the activity target determined to be similar to the long-term target for the target patient: and performing a process for presenting information about the extracted short-term target.

Further, a program according to the present disclosure causes a computer to perform: a function of storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients: a function of determining whether or not the activity target of each of the plurality of past patient is similar to a long-term target of the activity of a target patient: a function of extracting the short-term target set for the past patient corresponding to the activity target determined to be similar to the long-term target for the target patient: and a function of performing a process for presenting information about the extracted short-term target.

### Advantageous Effects of Invention

According to the present disclosure, it is possible to provide an information processing apparatus, a support method, and a program capable of effectively supporting the creation of a plan for activities aimed at improving abilities.

### BRIEF DESCRIPTION OF DRAWINGS

FIG. 1 shows an outline of an information processing apparatus according to an example embodiment of the present disclosure:

FIG. 2 is a flowchart showing a support method performed by an information processing apparatus according to an example embodiment of the present disclosure:

FIG. 3 shows a support system according to a first example embodiment:

FIG. 4 shows a configuration of an information processing apparatus according to the first example embodiment:

FIG. 5 is a flowchart showing a support method performed by the information processing apparatus according to the first example embodiment;

FIG. 6 is a table showing an example of past patient data according to the first example embodiment:

FIG. 7 is a table showing an example of a patient feature vector used for the calculation of the degree of similarity performed in a patient information determination unit according to the first example embodiment:

FIG. 8 shows a diagram for explaining processes in steps S102 to S106 performed by the information processing apparatus according to first example embodiment:

FIG. 9 is a diagram for explaining an example of a method for determining a long-term target for a target patient according to the first example embodiment:

FIG. 10 shows an example of a word feature vector used in the calculation of the degree of similarity performed by a long-term target determination unit according to the first example embodiment;

FIG. 11 shows a diagram for explaining processes in steps S112 to S116 performed by the information processing apparatus according to first example embodiment:

FIG. 12 shows a specific example of a method for extracting a short-term target according to the first example embodiment:

FIG. 13 shows an example of a method for determining a short-term target for a target patient according to the first example embodiment:

FIG. 14 shows an example of a display screen displayed on a user interface of an information processing apparatus or a user terminal according to the first example embodiment:

FIG. 15 is a flowchart showing a support method performed by the information processing apparatus according to a second example embodiment:

FIG. 16 shows an example of a word feature vector used in the calculation of the degree of similarity performed in a long-term target determination unit according to the second example embodiment:

FIG. 17 shows another example of the word feature vector used in the calculation of the degree of similarity performed in a long-term target determination unit according to the second example embodiment:

FIG. 18 shows a configuration of an information processing apparatus according to a third example embodiment:

FIG. 19 is a diagram for explaining processes performed by a keyword generation unit according to the third example embodiment:

FIG. 20 is a table showing an example of past patient data according to the third example embodiment: and

FIG. 21 shows an example of a patient feature vector used for the calculation of the degree of similarity performed in a patient information determination unit according to the third example embodiment.

### DESCRIPTION OF EMBODIMENTS

### Outline of Example Embodiment According to Present Disclosure

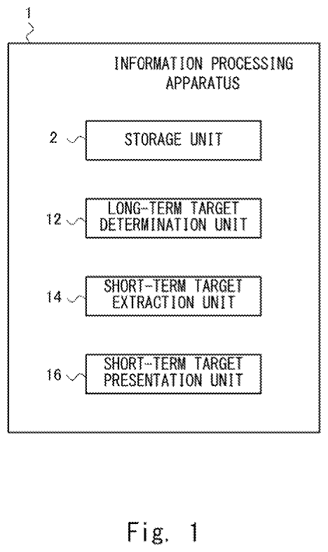

Prior to describing an example embodiment according to the present disclosure, an outline of the example embodiment will be described. FIG. 1 shows an outline of an information processing apparatus 1 according to an example embodiment of the present disclosure. The information processing apparatus 1 is, for example, a computer such as a server. The information processing apparatus 1 performs processes related to a target of activities aimed at improving abilities (ability improving activities) such as rehabilitation.

The information processing apparatus 1 includes a storage unit 2, a long-term target determination unit 12, a short-term target extraction unit 14, and a short-term target presentation unit 16. The storage unit 2 has a function as storage means. The long-term target determination unit 12 has a function as long-term target determination means. The short-term target extraction unit 14 has a function as short-term target extraction means. The short-term target presentation unit 16 has a function as short-term target presentation means.

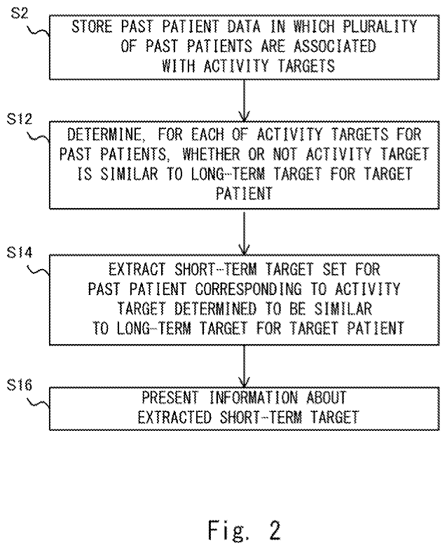

FIG. 2 is a flowchart showing a support method (an information processing method) performed by the information processing apparatus 1 according to an example embodiment of the present disclosure. The storage unit 2 stores therein past patient data in which a plurality of past patients are associated with activity targets (Step S2). Note that the “past patients” are patients who performed activities aimed at improving their abilities in the past. Further, the “activities aimed at improving abilities” are, for example, rehabilitation (e.g., a rehabilitation training). Further, each of the activity targets includes at least a short-term target for an activity(ies) (an ability improving activity(ies)) performed by a respective one of the plurality of past patients. The short-term target will be described later.

The long-term target determination unit 12 determines, for each of the plurality of past patients, whether or not the activity target for the past patient is similar to a long-term target for an activity(ies) (an ability improving activity(ies)) performed by a target patient (Step S12). The short-term target extraction unit 14 extracts short-term targets set for past patients corresponding to the activity targets which have been determined to be similar to the long-term target for the target patient (Step S14). The short-term target presentation unit 16 performs a process for presenting information about the extracted short-term targets to a user (Step S16).

Note that the “information about short-term targets” may be, for example, information indicating candidates for short-term targets that will be used as references for the user such as a therapist when he/she creates (determines) a plan for a short-term target. Alternatively, the “information about short-term targets” may be, for example, information indicating a short-term target that is determined by the information processing apparatus 1 or the like according to the extracted short-term targets. Further, the “process for presenting information to a user” may be a process for displaying the information on a terminal possessed (e.g., carried) by an individual user. Alternatively, the “process for presenting information to a user” may be a process for displaying the information on a display device (a user interface) provided in the information processing apparatus 1.

Since the information processing apparatus 1 according to the present disclosure is configured as described above, it can reduce the time for creating a plan for activities aimed at improving abilities without creating in advance a correspondence table in which long-term targets are associated with short-term targets. That is, the information processing apparatus 1 according to the present disclosure enables the user to easily create a plan for activities aimed at improving abilities. Therefore, the information processing apparatus 1 according to the present disclosure can make it possible to effectively support the creation of a plan for activities aimed at improving abilities. Note that it is also possible to effectively support the creation of a plan for activities aimed at improving abilities by using a support method performed by the information processing apparatus 1 or a program for performing a support method.

### First Example Embodiment

An example embodiment will be described hereinafter with reference to the drawings. For clarifying the explanation, the following description and the drawings have been partially omitted and simplified as appropriate. Further, the same symbols are assigned to the same or corresponding components throughout the drawings and redundant descriptions thereof are omitted as appropriate.

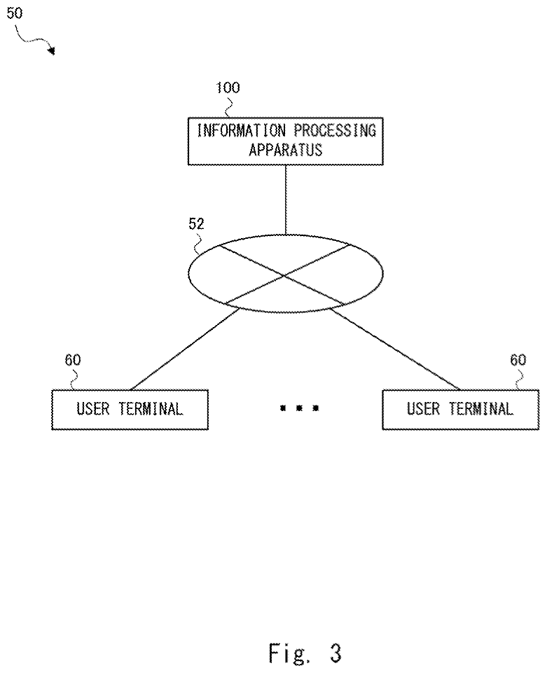

FIG. 3 shows a support system 50 according to a first example embodiment. The support system 50 includes at least one user terminal 60 and an information processing apparatus 100. The information processing apparatus 100 corresponds to the information processing apparatus 1 shown in FIG. 1. The user terminal 60 and the information processing apparatus 100 are connected to each other through a wired or wireless network 52 so that they can communicate with each other. The information processing apparatus 100 is, for example, a computer such as a server. The information processing apparatus 100 supports the creation of a plan for rehabilitation (an ability improving activity(ies)). More specifically, the information processing apparatus 100 performs a process for supporting the creation of a target (a long-term target and a short-term target) for rehabilitation. More specifically, the information processing apparatus 100 performs a process for presenting, to a user, information for supporting the creation of a target (a long-term target and a short-term target) for rehabilitation. The long-term and short-term targets will be described later.

The user terminal 60 is, for example, a computer. The user terminal 60 is, for example, a personal computer (PC), a tablet-type terminal, or a portable terminal such as a smartphone of a user such as a therapist. The user may input patient information, i.e., information about a patient by using the user terminal 60. In such a case, the user terminal 60 receives the patient information through an input device. Then, the user terminal 60 transmits the patient information to the information processing apparatus 100. The patient information indicates at least features of the patient. The patient information will be described later.

Further, the user terminal 60 may receive information about a long-term target and a short-term target through a process performed by the information processing apparatus 100. In such a case, the user terminal 60 displays the information received from the information processing apparatus 100 on a display device (a user interface) provided in the user terminal 60. Note that the information processing apparatus 100 may display information in the user interface provided in the information processing apparatus 100.

A rehabilitation plan (a rehab plan) will be described hereinafter. A therapist in a rehabilitation hospital often creates, when a patient is admitted to the hospital, a rehabilitation plan of the patient for the discharge from the hospital. Note that one of the important items in the rehabilitation plan is a long-term target and a short-term target.

The long-term target indicates the conditions of the patient and activities that the patient can perform, which are a target immediately before the discharge in the rehabilitation plan. That is, the long-term target indicates the conditions of the patient that are desired to be achieved at the time of discharge, and also indicates the conditions of the patient and movements that the patient can perform both of which are required to enable the patient to be discharged from the hospital to a place where the patient wishes to stay after the discharge. Meanwhile, the short-term target indicates the conditions of the patient and movements that the patient can perform both of which are a target at a midpoint in the planned hospitalization period in the rehabilitation plan. That is, the short-term target indicates the conditions of the patient and movements that the patient can perform both of which should be achieved in order to achieve the long-term target. Note that the timing of the short-term target is often determined for each patient in each facility in a flexible manner, and may be a timing that is determined as appropriate when the rehabilitation plan is made. For example, the short-term target may be set on a weekly basis or at a midpoint in the planned hospitalization period.

Note that, in a rehabilitation hospital, for example, therapists of three types professions, i.e., a physical therapist (PT: Physical Therapist), an occupational therapist (OT: Occupational Therapist), and speech-language-hearing therapist (ST: Speech-Language-Hearing Therapist), are in charge of one patient. Further, these therapists of three types professions discuss and set a short-term target and a long-term target.

Note that, in many cases, each of the physical therapist, the occupational therapist, and the speech-language-hearing therapist is in charge of many patients. Therefore, the time when all of these therapists of three types professions can meet is limited. Therefore, it is very troublesome for the therapists of three types professions to meet and discuss for about 30 minutes. Therefore, a method by which a long-term target and a short-term target can be determined in a short time has been desired. Meanwhile, the support system 50 and the information processing apparatus 100 according to this example embodiment can assist a user (a therapist) to determine a long-term target and a short-term target in a short time as will be described hereinafter.

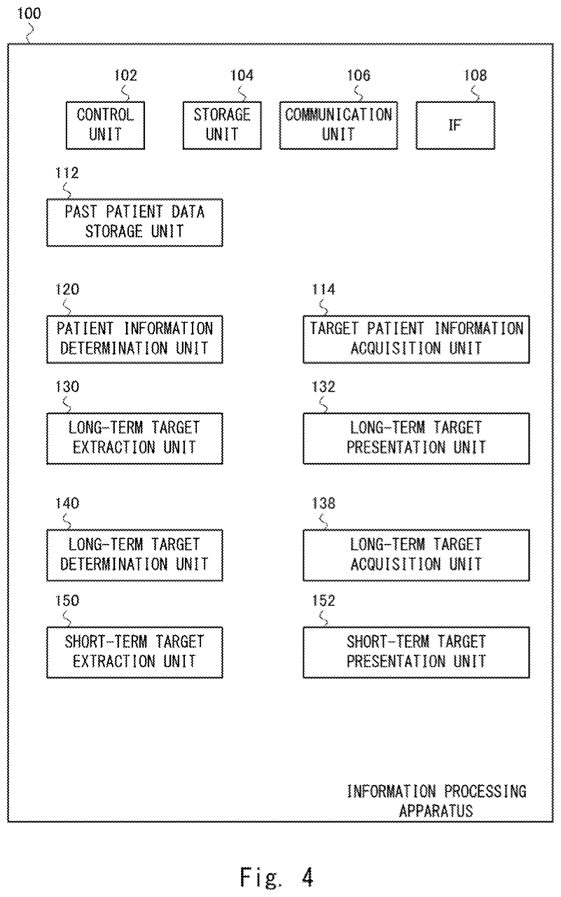

FIG. 4 shows a configuration of the information processing apparatus 100 according to the first example embodiment. The information processing apparatus 100 includes, as its main hardware configuration, a control unit 102, a storage unit 104, a communication unit 106, and an interface (IF: Interface) unit 108. The control unit 102, the storage unit 104, the communication unit 106, and the interface unit 108 are connected to each other through a data bus or the like. Note that the user terminal 60 shown in FIG. 3 can also have the hardware configuration shown in FIG. 4.

The control unit 102 is a processor such as a CPU (Central Processing Unit). The control unit 102 has a function as an arithmetic apparatus that performs control processing, arithmetic processing, and the like. The storage unit 104 is, for example, a memory or a storage device such as a hard disk drive. The storage unit 104 is, for example, a ROM (Read Only Memory), a RAM (Random Access Memory) or the like. The storage unit 104 has a function of storing a control program(s), an arithmetic program(s), and the like performed by the control unit 102. Further, the storage unit 104 has a function of temporarily storing processing data and the like. The storage unit 104 can include a database.

The communication unit 106 performs processing necessary to communicate with the user terminal 60 (and other devices) through a network 52. The communication unit 106 can include communication ports, a router, a firewall, and the like. The interface (IF: Interface) unit 108 is, for example, a user interface (UI). The interface unit 108 includes an input device such as a keyboard, a touch panel, or a mouse, and an output device such as a display or a speaker(s). The interface unit 108 receives data input by a user (an operator) and outputs information for the user. The interface unit 108 may display information about a long-term target and a short-term target.

The information processing apparatus 100 according to the first example embodiment includes, as its components, a past patient data storage unit 112, a target patient information acquisition unit 114, a patient information determination unit 120, a long-term target extraction unit 130, and a long-term target presentation unit 132. Further, the information processing apparatus 100 according to the first example embodiment includes, as its components, a long-term target acquisition unit 138, a long-term target determination unit 140, a short-term target extraction unit 150, and a short-term target presentation unit 152.

The past patient data storage unit 112 corresponds to the storage unit 2 shown in FIG. 1. The past patient data storage unit 112 has a function as past patient data storage means. The target patient information acquisition unit 114 has a function as target patient information acquisition means. The patient information determination unit 120 has a function as patient information determination means. The long-term target extraction unit 130 has a function as long-term target extraction means. The long-term target presentation unit 132 has a function as long-term target presentation means. The long-term target acquisition unit 138 has a function as long-term target acquisition means.

The long-term target determination unit 140 corresponds to the long-term target determination unit 12 shown in FIG. 1. The long-term target determination unit 140 has a function as long-term target determination means. The short-term target extraction unit 150 corresponds to the short-term target extraction unit 14 shown in FIG. 1. The short-term target extraction unit 150 has a function as short-term target extraction means. The short-term target presentation unit 152 corresponds to the short-term target presentation unit 16 shown in FIG. 1. The short-term target presentation unit 152 has a function as short-term target presentation means.

Note that each of the above-described components can be implemented, for example, by executing a program under the control of the control unit 102. More specifically, each of the components can be implemented by having the control unit 102 execute a program stored in the storage unit 104. Further, each of the components can be implemented by recording a necessary program on an arbitrary non-volatile recording medium and installing it as required. Further, each of the components is not limited to those implemented by software using a program, and may be implemented by a combination of any two or more of hardware, firmware, and software. Further, each of the components may be implemented by using a user-programmable integrated circuit such as an FPGA (Field-Programmable Gate Array) or a microcomputer. In such a case, a program composed of a respective one of the above-described components may be implemented by using this integrated circuit. The above-described matters also apply to other example embodiments (which will be described later). Note that the specific function of each of the components will be described later.

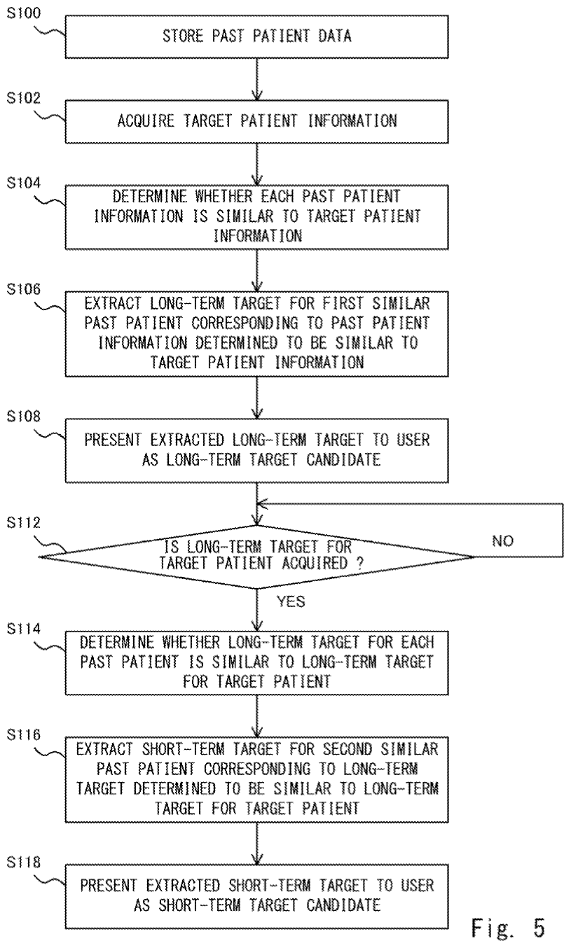

FIG. 5 is a flowchart showing a support method performed by the information processing apparatus 100 according to the first example embodiment.

The past patient data storage unit 112 stores past patient data related to past patients who performed rehabilitation in the past (Step S100). In the past patient data, a plurality of past patients, a plurality of pieces of patient information for the respective past patients, and a plurality of long-term targets and a plurality of short-term targets for the respective past patients are associated with each other.

The past patient data storage unit 112 may, for example, extract past patient data from electronic medical records and store the extracted past patient data in a database (the storage unit 104). Note that the patient information is information indicating at least features of the corresponding patient.

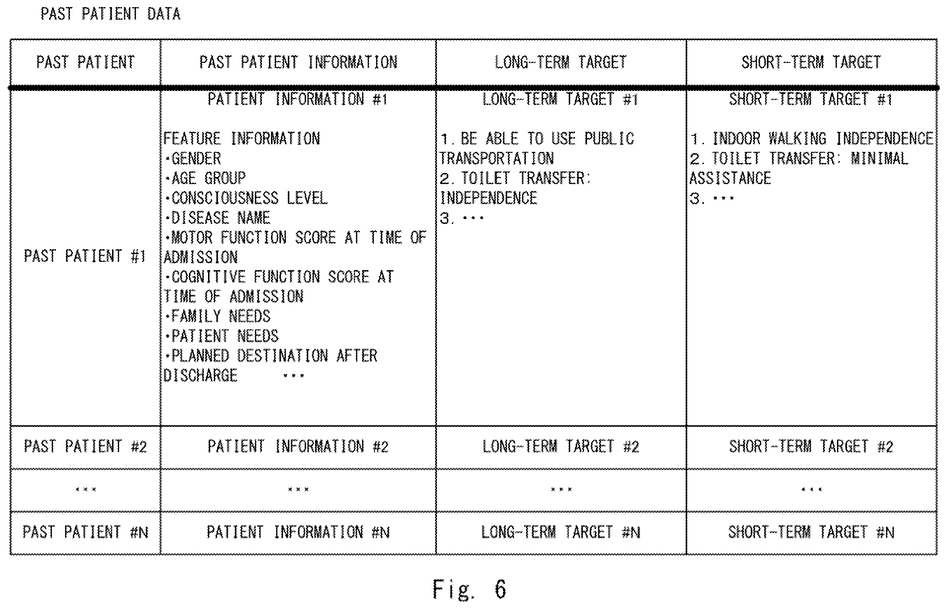

FIG. 6 is a table showing an example of past patient data according to the first example embodiment. In the example shown in FIG. 6, in the past patient data, past patients, pieces of past patient information, long-term targets, and short-term targets are associated with each other. For example, in the past patient data, a past patient #1, patient information #1, which is past patient information about the past patient #1, a long-term target #1 for the past patient #1, and a short-term target #1 for the past patient #1 are associated with each other. Further, in the past patient data, a past patient #N, patient information #N, which is past patient information about the past patient #N, a long-term target #N for the past patient #N, and a short-term target #N for the past patient #N are associated with each other. That is, in the past patient data, long-term targets #1 to #N and short-term targets #1 to #N are set for the past patients #1 to #N, respectively.

In the first example embodiment, the patient information includes feature information indicating at least features (feature values) of the patient. The patient information (the feature information) can include, for example, patient's attributes (a gender, an age group, etc.), a consciousness level, a disease name, a motor function score at the time of admission, a cognitive function score at the time of admission, family needs, patient needs, and a planned destination after the discharge. However, these items are merely examples, and the items in the patient information (the feature information) are not limited to these items. For example, the patient information may include terms related to the individual circumstances of the patient.

Note that the “consciousness level” may be represented, for example, by a score according to GCS (Glasgow Coma Scale) or JCS (Japan Coma Scale), or may be represented by a term based on the user's point of view. Further, the “motor function score” and the “cognitive function score” (hereinafter, they may also be collectively referred to as the “function score(s)”) are, for example, ability values (ability levels) related to patient's activities in daily life. The function score can be, for example, a score (an index) related to ADL (Activities of Daily Living) or IADL (Instrumental Activities of Daily Living). For example, the function score is an evaluation score for a respective evaluation item included in an FIM (Function Independence Measure) table. However, other types of scores may also be used.

Further, the “family needs” and the “patient needs” (hereinafter, they may also be collectively referred to as simply “needs”) are, for example, needs of the patient's family and those of the patient himself/herself in the rehabilitation plan. For example, the needs may be a target in the rehabilitation plan (e.g., abilities or the like that the patient wishes to have at the time of discharge). Further, the “planned destination after discharge” may include information about the type of the building where the patient will stay after the discharge (e.g., whether the building is a condominium or a two-story detached house), information about the presence/absence of a roommate(s), and information about transportation facilities in the place where the patient will stay after the discharge.

Further, the long-term target in the past patient data is a long-term target that has been actually created for the corresponding past patient. Similarly, the short-term target in the past patient data is a short-term target that has been actually created for the corresponding past patient. Note that the long-term and short-term targets in the past patient data can be those that have been created while taking the points of view of the three types of professions, e.g., a physical therapist, an occupational therapist, and a speech-language-hearing therapist into consideration. Therefore, the long-term and short-term targets in the past patient data can be suitable for the corresponding past patient.

In FIG. 6, for example, the long-term target #1 actually created for the past patient #1 may include an item “Be able to use public transportation”, and an item composed of an evaluation item “Toilet transfer” and the degree of independence “Independence”. Further, the short-term target #1 actually created for the past patient #1 may include an item “Indoor walking independence”, and an item composed of an evaluation item “Toilet transfer” and the degree of independence “Maximum assistance”. Note that the degree of independence may be, for example, an ability value (an ability level). The degree of independence may be, for example, a score in the FIM specified as shown below.
7 points: Complete independence (including a time and safety)
6 points: Modified independence (independence with use of a help device)
5 points: Supervision (instruction, prompting, and preparation are required)
4 points: Minimal assistance (25% or lower assistance is required)
3 points: Moderate assistance (25% to 50% assistance is required)
2 points: Maximum assistance (50% to 75% assistance is required)
1 point: Total assistance (75% or higher assistance is required)

Further, the evaluation items may be the below-shown items specified in the FIM.

Cognitive items: comprehension, expression, social interaction, problem solving, and memory.
Motor items: eating, dressing, wiping, changing (upper body), changing (lower body), toileting, urinary management, defecation management, bed/wheelchair/chair transfer, toilet transfer, bath/shower transfer, walking/wheelchair transfer, and stair transfer.

Note that, as shown in FIG. 6, the long-term and short-term targets may include items other than those composed of evaluation items and the degrees of independence. Further, in addition to the items shown in FIG. 6, the long-term and short-term targets may include, for example, an item “Be able to eat regular food” and an item “Be able to leave bed for one hour or longer”. That is, the long-term and short-term targets can be, for example, described by the user such as a therapist in an arbitrary manner.

The target patient information acquisition unit 114 acquires patient information of a target patient (target patient information) (Step S102). Specifically, the target patient information acquisition unit 114 may, for example, acquire (receive) target patient information from the user terminal 60 through the network 52 and the communication unit 106. Alternatively, the target patient information acquisition unit 114 may acquire target patient information that is input by using the interface unit 108. Note that the target patient information can include feature information (feature values) similar to the feature information of the past patient information shown in FIG. 6.

The patient information determination unit 120 determines whether or not each of a plurality pieces of past patient information is similar to the target patient information (Step S104). That is, the patient information determination unit 120 determines, for each of the plurality of past patients, whether or not the past patient information of the past patient is similar to the target patient information indicating at least features of the target patient. Specifically, the patient information determination unit 120 calculates the degree of similarity between each of the plurality pieces of past patient information and the target patient information. Then, the patient information determination unit 120 may determine that pieces of past patient information of which the degrees of similarity are larger than or equal to a predetermined threshold are similar to the target patient information. Alternatively, the patient information determination unit 120 may determine that pieces of past patient information of which the degrees of similarity are within the top Nc1 ranks (Nc1 is a predetermined integer greater than or equal to one) are similar to the target patient information.

Various methods can be adopted for the method for calculating the degree of similarity. For example, as described later, the degree of similarity between target patient information and past patient information may be calculated by calculating a distance (a Euclidean distance) between a feature vector corresponding to the target patient information and a feature vector corresponding to the past patient information. In such a case, it means that the smaller the distance between the feature vectors is, the higher the degree of similarity between the corresponding target patient information and the corresponding past patient information is. Note that the aforementioned distance may be calculated while assigning a predetermined weight to each element of the feature vector (i.e., to each item in the past patient information and the target patient information). However, the above-described calculation method is merely an example, and any other calculation method may be used to determine the degree of similarity between features of the target patient and those of the past patient. For example, after manually repeating matching processes between the past patient information and the target patient information, and thereby training a model through machine learning such as deep learning, the degree of similarity between the past patient information and the target patient information may be calculated by using the learning result (i.e., by using the trained model).

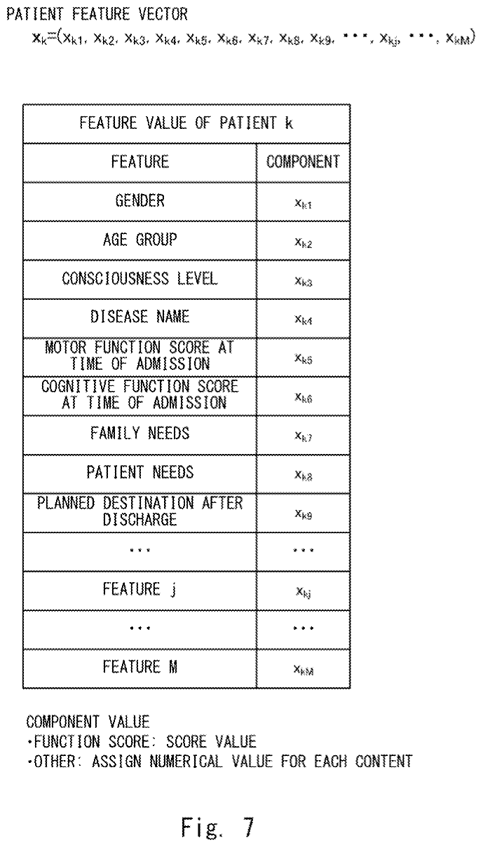

FIG. 7 is a table showing an example of a patient feature vector used for the calculation of the degree of similarity performed in the patient information determination unit 120 according to the first example embodiment. The patient feature vector indicates feature values of the above-described patient information (the target patient information and the past patient information). Note that the patient feature vector shown in FIG. 7 is merely an example, and various other patient feature vectors can be used. FIG. 7 shows an example of a patient feature vector of a past patient, and the patient feature vector of the target patient is similar to this example as described later. Here, the total number of past patients is represented by N (N is an integer greater than or equal to two) and the number of features is represented by M (M is an integer greater than or equal to one). Then, when a patient feature vector of a patient k (k is an integer from 1 to N) is represented by xₖ, the M components of the feature vector are expressed by the below-shown Expression 1.

xₖ=(xₖ₁, xₖ₂, xₖ₃, xₖ₄, xₖ₅, xₖ₆, xₖ₇, xₖ₈, xₖ₉, . . . , xₖⱼ, . . . , x_{kM}) (1)

where j is an integer no smaller than one and no greater than M, and is an index indicating a feature.

In the example shown in FIG. 7, for example, xₖ₁ represents the gender of the patient k (the past patient k). Further, xₖ₂ represents an age group of the patient k. Further, xₖ₅ represents a motor function score of the patient k at the time of admission. Further, xₖ₆ represents a cognitive function score of the patient k at the time of admission. Note that the number of components M of the patient feature vector, and what kind of feature each component value indicates may be determined by the user in advance.

Note that when a feature is represented by a numerical value such as a score, a component value xₖⱼ can indicate a score value. For example, component values xₖ₅ and xₖ₆ may indicate score values of the respective function scores (such as FIM values). Further, when a feature is not represented by a numerical value, a component value xₖⱼ can be assigned a numerical value for a respective content. For example, in the case of the feature “gender,” the component value xₖ₁ is assigned “1” (xₖ₁=1) when the patient is “male”, and the component value xₖ₁ is assigned “0” (xₖ₁=0) when the patient is “female.

Further, a patient feature vector xᵢ of a target patient i is expressed by the below-shown Expression 2 as in the case of the Expression 1.

xᵢ=(xᵢ₁, xᵢ₂, xᵢ₃, xᵢ₄, xᵢ₅, xᵢ₆, xᵢ₇, xᵢ₈, xᵢ₉, . . . ,xᵢⱼ, . . . , x_{iM}) (2)

The patient information determination unit 120 calculates, for example, the degree of similarity Dpᵢₖ between the patient information of the target patient i (the target patient information) and the patient information of the past patient k (the past patient information) as shown in the below-shown Expressions 3 and 4.

$${Dp}_{ik} = 1 / {Sp}_{ik} \qquad ( 3 )$$

$$\begin{matrix}[ Expression 1 ] & \\ {Sp}_{ik} = \sqrt{{\sum }_{j = 1}^{M} {( {x}_{ij} - {x}_{kj} )}^{2}} & ( 4 )\end{matrix}$$

The long-term target extraction unit 130 extracts long-term targets for past patients (first similar past patients) corresponding to the pieces of past patient information which have been determined to be similar to the target patient information in the process in the step S104 (Step S106). That is, the long-term target extraction unit 130 extracts long-term targets set for past patients corresponding to the pieces of past patient information which have been determined to be similar to the target patient information. Specifically, the long-term target extraction unit 130 extracts, by using the past patient data shown in FIG. 6, long-term targets for first similar past patients corresponding to the pieces of past patient information which have been determined to be similar to the target patient information.

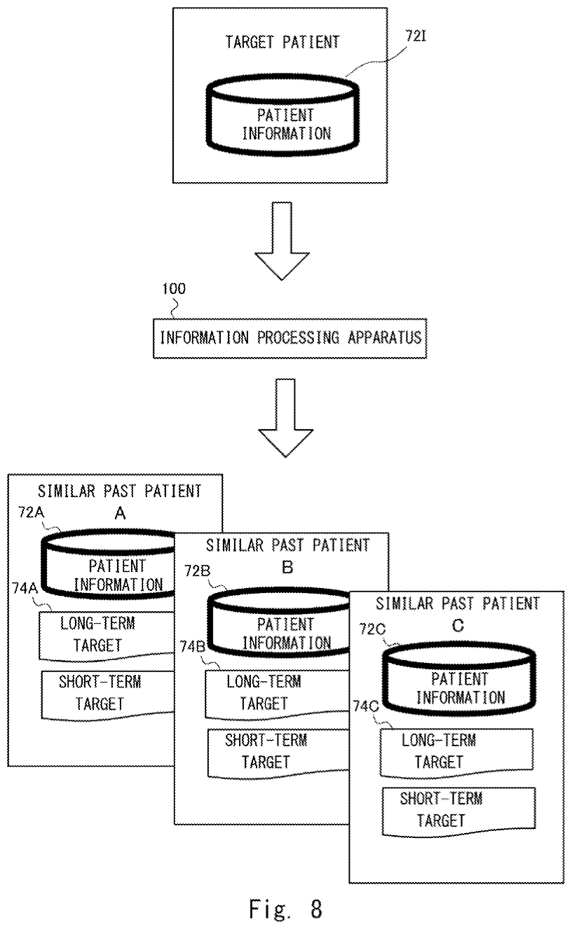

FIG. 8 shows a diagram for explaining the processes in the steps S102 to S106 performed by the information processing apparatus 100 according to the first example embodiment. When patient information 72I of the target patient is input to the information processing apparatus 100 (S102), the patient information determination unit 120 of the information processing apparatus 100 determines pieces of patient information (pieces of past patient information) that are similar to the patient information 72I (S104). In the example shown in FIG. 8, the patient information determination unit 120 determines that each of patient information 72A of a similar past patient A, patient information 72B of a similar past patient B, and patient information 72C of a similar past patient C is similar to the patient information 72I. The long-term target extraction unit 130 extracts a long-term target 74A corresponding to the patient information 72A, a long-term target 74B corresponding to the patient information 72B, and a long-term target 74C corresponding to the patient information 72C.

The long-term target presentation unit 132 performs a process for presenting the long-term targets extracted in the process in the step S106 to the user as candidates for the long-term target for the target patient (long-term target candidates) (Step S108). That is, the long-term target presentation unit 132 performs a process for presenting information about the extracted long-term targets. Specifically, the long-term target presentation unit 132 performs a process for displaying long-term target candidates on a display device (an output device) of the user terminal 60 by controlling the communication unit 106. Alternatively, the long-term target presentation unit 132 performs a process for displaying long-term target candidates on the interface unit 108 of the information processing apparatus 100. Note that the long-term target presentation unit 132 does not need to present all the long-term targets extracted in the process in the step S106 to the user as the long-term target candidates. The long-term target presentation unit 132 may present, among the extracted long-term targets, a predetermined number of long-term targets that are selected in descending order of the degrees of similarity of the corresponding pieces of past patient information to the user as the long-term target candidates.

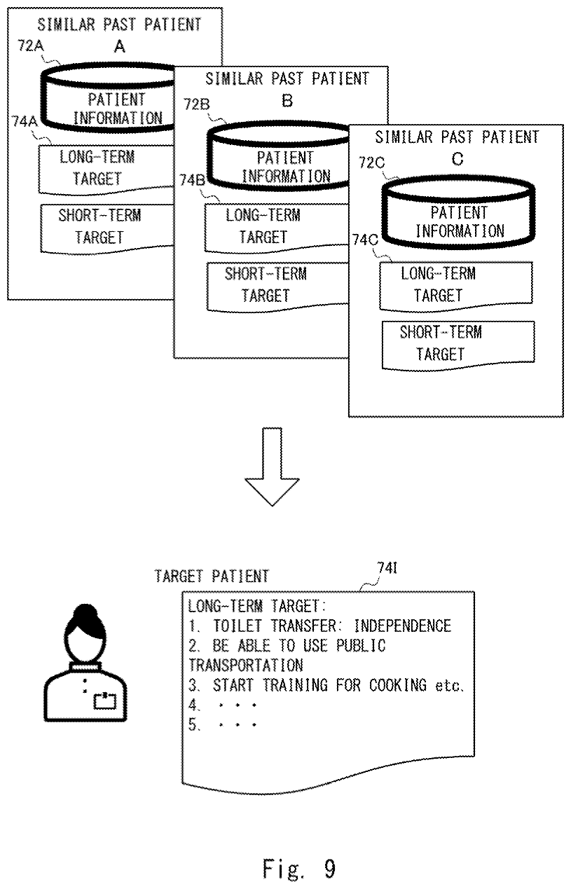

FIG. 9 is a diagram for explaining an example of a method for determining a long-term target for a target patient according to the first example embodiment.

The long-term target presentation unit 132 of the information processing apparatus 100 presents the long-term targets 74A, 74B and 74C of the similar past patients A, B and C to the user as the long-term target candidates. The user determines the long-term target 74I for the target patient by referring to these long-term target candidates. For example, the user may determine the long-term target for which the degree of similarity of the corresponding past patient information is the highest (e.g., the long-term target 74A) as the long-term target 74I for the target patient. Alternatively, for example, the user may determine the long-term target 74I by arbitrarily selecting items of the presented long-term targets 74A to 74C. The user inputs the determined long-term target 74I to the information processing apparatus 100 by using the interface unit 108.

As described above, the information processing apparatus 100 according to the first example embodiment determines, for each of a plurality pieces of past patient information, whether or not the piece of past patient information is similar to the target patient information. Then, the information processing apparatus 100 according to the first example embodiment extracts long-term targets corresponding to the pieces of past patient information which have been determined to be similar to the target patient information, and performs a process for presenting the extracted long-term targets as long-term target candidates for the target patient.

Note that, as described above, the long-term targets in the past patient data can be those that have been created while taking the points of view of the three types of professions, e.g., a physical therapist, an occupational therapist, and a speech-language-hearing therapist into consideration. As a result, the extracted long-term targets can be those that have been created while taking the points of view of the three types of professions into consideration. Therefore, by presenting the extracted long-term targets as long-term target candidates for the target patient, it is possible to easily determine a long-term target in which the points of view of the three types of professions are taken into consideration. That is, when the above-described three types of professions meet and discuss the long-term target, they can select a long-term target from a limited number of long-term target candidates that are presented while taking the points of view of the three types of professions into consideration (or can determine a long-term target by using a limited number of long-term target candidates). Therefore, it is possible to determine a long-term target in a shorter time than in the case where long-term targets suitable for the target patient are examined one by one from among a myriad of long-term target candidates (which are not presented to the user). Therefore, the user (a therapist) can determine the long-term target in a short time.

Further, the information processing apparatus 100 according to the first example embodiment performs a process for presenting the extracted long-term targets as the long-term target candidates for the target patient. In this way, the user (a therapist or the like) can easily and appropriately create a long-term target by referring to the presented long-term target candidates. Note that, instead of having the user determine the long-term target for the target patient, the information processing apparatus 100 may determine a long-term target corresponding to past patient information of which the degree of similarity is the highest as the long-term target for the target patient. However, in the case where the information processing apparatus 100 determines the long-term target, there is a possibility that the individual circumstances of the target patient are not taken into consideration in the determined long-term target. Therefore, by presenting the extracted long-term targets as the long-term target candidates for the target patient, the user can create a long-term target in which the individual circumstances of the target patient are taken into consideration, so that a more appropriate long-term target can be created.

The long-term target acquisition unit 138 acquires the long-term target 74I for the target patient through the interface unit 108. When the long-term target acquisition unit 138 acquires the long-term target 74I for the target patient (Yes in Step S112), the long-term target determination unit 140 determines whether or not the long-term target of each of the plurality of past patients is similar to the long-term target of the target patient (Step S114). That is, the long-term target determination unit 140 determines whether or not the activity target (the long-term target) of each of the plurality of past patients is similar to the long-term target for the activities (the rehabilitation) performed by the target patient. Specifically, the long-term target determination unit 140 determines whether or not a word(s) (a character string(s)) included in the activity target (the long-term target) of each of the plurality of past patients is similar to a word(s) (a character string(s)) included in the long-term target of the target patient.

The long-term target determination unit 140 calculates the degree of similarity between each of the long-term targets #1 to #N of the plurality of past patients and the long-term target 74I for the target patient. Then, the long-term target determination unit 140 may determine that long-term targets of which the degree of similarity is larger than or equal to a predetermined threshold are similar to the long-term target 74I for the target patient. Alternatively, the long-term target determination unit 140 may determine that long-term targets for past patients of which the degrees of similarity are within the top Nc2 ranks (Nc2 is a predetermined integer greater than or equal to one) are similar to the long-term target 74I for the target patient. The method for calculating the degree of similarity may be substantially the same as that in the step S104. However, the feature vectors used in the process in the step S114 are different from those used in the process in the step S104.

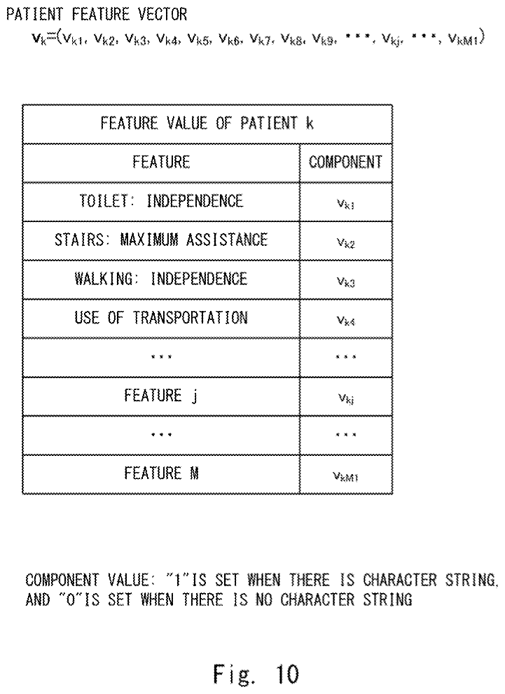

FIG. 10 shows an example of a word feature vector used in the calculation of the degree of similarity performed by the long-term target determination unit 140 according to the first example embodiment. The long-term target determination unit 140 determines, by using this word feature vector, whether or not a word(s) (a character string(s)) included in the long-term target (the activity target) of each of the plurality of past patients is similar to a word(s) (a character string(s)) included in the long-term target 74I for the target patient. Note that, in the first example embodiment, the components of the word feature vector can be, for example, character strings indicating the items in the long-term target. In particular, when each item in the long-term target is a pair of an evaluation item and the degree of independence, each component of the word feature vector can be a character string composed of a pair of an evaluation item and the degree of independence (an ability level).

The word feature vector shown in FIG. 10 indicates feature values in the long-term target (the long-term target for the target patient and long-term targets for past patients). Note that the word feature vector is merely an example, and various other word feature vectors can be used. Here, the total number of past patients is represented by N and the number of features is represented by M1 (M1 is an integer greater than or equal to one). Then, when a word feature vector of a patient k (a past patient k) is represented by vₖ, the MI components of the feature vector are expressed by the below-shown Expression 5.

vₖ=(vₖ₁, vₖ₂, vₖ₃, vₖ₄, vₖ₅, vₖ₆, vₖ₇, vₖ₈, vₖ₉, . . . , vₖⱼ, . . . , v_{kM1}) (5)

In the example shown in FIG. 10, for example, a component vₖ₁ corresponds to a character string “Toilet: Independence” (a pair of an evaluation item and the degree of independence) in the long-term target for a patient k. A component vₖ₂ corresponds to a character string “Stairs: Maximum assistance” (a pair of an evaluation item and the degree of independence) in the long-term target for the patient k. A component vₖ₃ corresponds to a character string “Walking: Independence” (a pair of an evaluation item and the degree of independence) in the long-term target for the patient k. A component vₖ₄ corresponds to a character string (an item) “Use of transportation” in the long-term target for the patient k. Note that the number of components M1 of the word feature vector, and what kind of feature each component value indicates may be determined by the user in advance. Further, the degree of independence may also be indicated by the above-described FIM value (the FIM score).

Note that the component value of each component of the word feature vector can be assigned depending on whether or not the corresponding word (or a character string similar to the corresponding word) is present in the long-term target. For example, when a character string “Toilet: Independence” is present in the long-term target for the patient k, the component value vₖ₁ is assigned “1” (vₖ₁=1), and when the character string “Toilet: Independence” is not present in the long-term target for the patient k, the component value vₖ₁ is assigned “0” (vₖ₁=0). Note that when at least one of character strings “Toilet transfer: Independence” and “Toileting: Independence” is present in the long-term target for the patient k, the component value vₖ₁ can be assigned “1” (vₖ₁=1). That is, when there is an evaluation item including a character string “Toilet” in the long-term target and the degree of independence thereof includes “Independence”, the component value vₖ₁ can be assigned “1” (vₖ₁=1). Further, for example, when a character string “Use of public transportation” is present in the long-term target for the patient k, the component value vₖ₄ is assigned “1” (vₖ₄=1) because the character string “Use of public transportation” is similar the character string “Use of transportation”. This feature also applies to the other components. Note that the determination of similarity between character strings may be performed by using, for example, a pre-stored similarity dictionary or a machine learning algorithm.

Further, the component value of each component of the word feature vector may be assigned the degree of independence (an FIM value) in the corresponding evaluation item. For example, when there is an evaluation item including a character string “Toilet” in the long-term target for the patient k and its degree of independence is “Complete independence”, the component value vₖ₁ can be assigned “7” (vₖ₁=7). Further, for example, when there is an evaluation item including a character string “Toilet” in the long-term target for the patient k and its degree of independence is “Modified independence”, the component value vₖ₁ can be assigned “6” (vₖ₁=6). Further, for example, when there is an evaluation item including a character string “Stairs” in the long-term target for the patient k, and its degree of independence is “Maximum assistance”, the component value vₖ₁ can be assigned “2” (vₖ₂=2). In this case, when there is no corresponding evaluation item in the long-term target for the patient k, its component value can be assigned “0”. These features also apply to the other components. Note that a dictionary (a degree-of-independence dictionary) in which character strings indicating degrees of independence are associated with levels (numerical values) corresponding to the degrees of independence may be stored in advance, and component values may be assigned numerical values by using this degree-of-independence dictionary. Further, the degree of independence does not have to be expressed by a character sting, and instead may be expressed by a numerical value indicating its level.

Further, a word feature vector vᵢ of a target patient i is expressed by the below-shown Expression 6 as in the case of the Expression 5.

vᵢ=(vᵢ₁, vᵢ₂, vᵢ₃, vᵢ₄, vᵢ₅, vᵢ₆, vᵢ₇, vᵢ₈, vᵢ₉, . . . , vᵢⱼ, . . . , v_{iM1}) (6)

The long-term target determination unit 140 calculates, for example, the degree of similarity Dwᵢₖ between the long-term target for the target patient i and the long-term target for the past patient k by the below-shown Expressions 7 and 8.

$${Dw}_{ik} = 1 / {Sw}_{ik} \qquad ( 7 )$$

$$\begin{matrix}[ Expression 2 ] & \\ {Sw}_{ik} = \sqrt{{\sum }_{j = 1}^{M 1} {( {v}_{ij} - {v}_{kj} )}^{2}} & ( 8 )\end{matrix}$$

The short-term target extraction unit 150 extracts short-term targets of past patients (second similar past patients) corresponding to the long-term targets that have been determined to be similar to the long-term target for the target patient in the process in the step S114 (Step S116). That is, the short-term target extraction unit 150 extracts short-term targets set for past patients corresponding to the activity targets (the long-term targets) that have been determined to be similar to the long-term target for the target patient. Specifically, the short-term target extraction unit 150 extracts short-term targets for second similar past patients corresponding to the long-term targets that have been determined to be similar to the long-term target for the target patient by using the past patient data shown in FIG. 6.

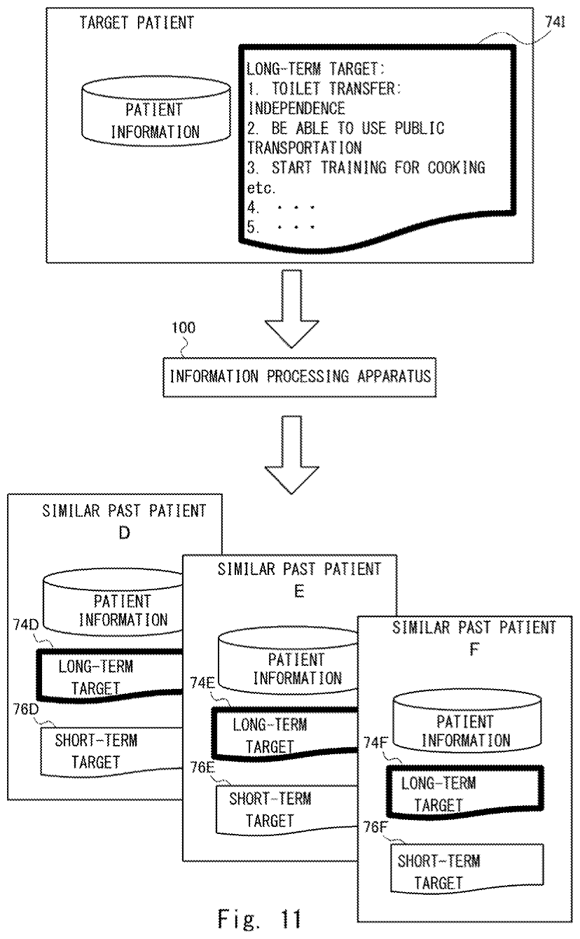

FIG. 11 shows a diagram for explaining the processes in the steps S112 to S116 performed by the information processing apparatus 100 according to the first example embodiment. When the long-term target 74I for the target patient is input to the information processing apparatus 100 (Yes in Step S112), the long-term target determination unit 140 of the information processing apparatus 100 determines long-term targets of past patients that are similar to the long-term target 74I (S114). In the example shown in FIG. 11, the long-term target determination unit 140 determines that a long-term target 74D of a similar past patient D, a long-term target 74E of a similar past patient E, and a long-term target 74F of a similar past patient F are similar to the long-term target 74I. The short-term target extraction unit 150 extracts a short-term target 76D corresponding to the long-term target 74D, a short-term target 76E corresponding to the long-term target 74E, and a short-term target 76F corresponding to the long-term target 74F.

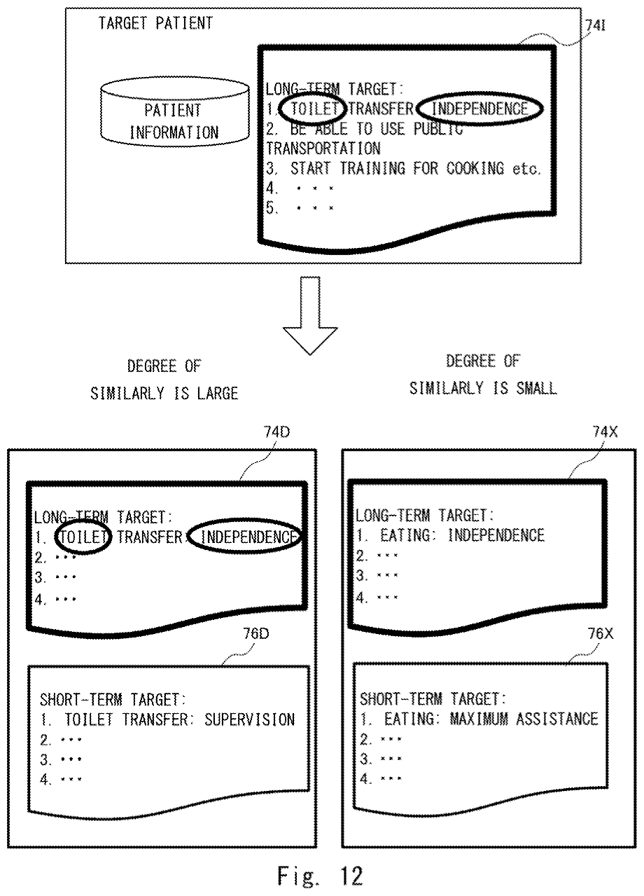

FIG. 12 shows a specific example of a method for extracting a short-term target according to the first example embodiment. An item “Toileting: Independence” is included in the long-term target 74I for the target patient.

Further, an item “Toilet transfer: Independence” is included in the long-term target 74D of the past patient. In this case, the character string “Toilet Independence” in the long-term target 74I is the same as the character string “Toilet Independence” in the long-term target 74D. Therefore, the long-term target determination unit 140 determines that the degree of similarity between the long-term target 74I and the long-term target 74D is large. Accordingly, the short-term target extraction unit 150 extracts the short-term target 76D corresponding to the long-term target 74D.

Meanwhile, the character string of any of the items included in the long-term target 74I for the target patient is not included in a long-term target 74X for a past patient. Therefore, the long-term target determination unit 140 determines that the degree of similarity between the long-term target 74I and the long-term target 74X is small. Therefore, the short-term target extraction unit 150 does not extract the short-term target 76X corresponding to the long-term target 74X. By determining whether or not a character string included in the long-term target (the activity target) of each of a plurality of past patients is similar to a character string included in the long-term target for the target patient, it is possible to determine long-term targets for past patients that are similar to the long-term target for the target patient in a more appropriate manner.

The short-term target presentation unit 152 performs a process for presenting, to the user, the short-term targets extracted in the process in the step S116 as candidates for the short-term target for the target patient (short-term target candidates) (Step S118). That is, the short-term target presentation unit 152 performs a process for presenting information about the extracted short-term targets. Specifically, the short-term target presentation unit 152 performs, by controlling the communication unit 106, a process for displaying the short-term target candidates on the display device (the output device) of the user terminal 60. Alternatively, the short-term target presentation unit 152 performs a process for displaying the short-term target candidates in the interface unit 108 of the information processing apparatus 100. Note that the short-term target presentation unit 152 does not need to present all the short-term targets extracted in the process in the step S116 to the user as the short-term target candidates. Among the extracted short-term targets, the short-term target presentation unit 152 may present, to the user, a predetermined number of short-term targets of which the degrees of similarity of the corresponding long-term targets are ranked higher than the others as the short-term target candidates.

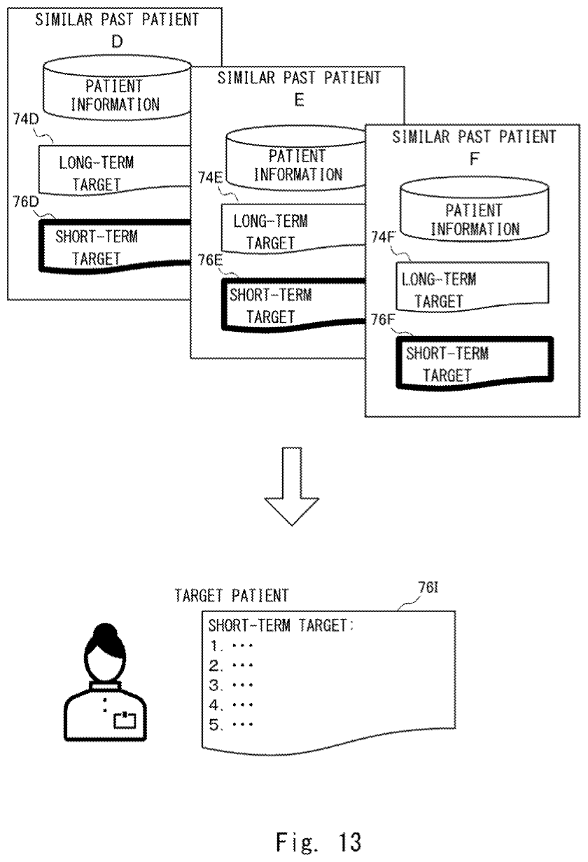

FIG. 13 shows an example of a method for determining a short-term target for a target patient according to the first example embodiment. The short-term target presentation unit 152 of the information processing apparatus 100 presents the short-term targets 76D, 76E and 76F of the similar past patients D, E and F to the user as short-term target candidates. The user determines the short-term target 76I for the target patient by referring to these short-term target candidates. For example, the user may use the short-term target of which the degree of similarity of the corresponding long-term target is the highest (e.g., the short-term target 76D) as it is as the short-term target 76I for the target patient. Alternatively, for example, the user may determine the short-term target 76I by arbitrarily selecting items in the presented short-term targets 76D to 76F.

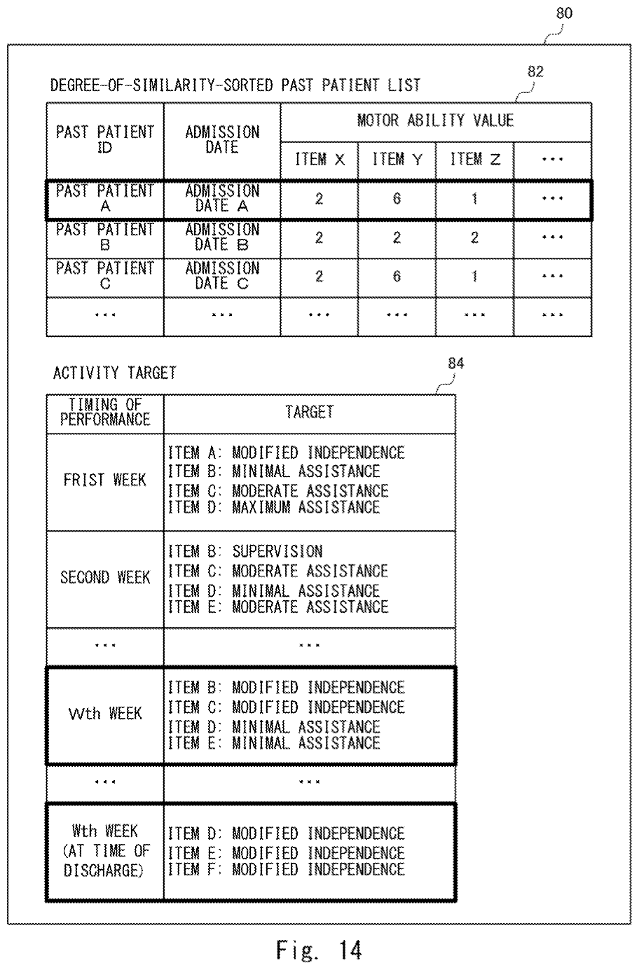

FIG. 14 shows an example of a display screen (e.g., a displayed window) 80 displayed in the user interface of the information processing apparatus 100 or the user terminal 60 according to the first example embodiment. The display screen 80 shown in FIG. 14 may be applied in either the process in the step S108 or the process in the step S118. The display screen 80 shows a list 82 of past patients sorted in the descending order of the similarity (hereinafter also referred to as a degree-of-similarity-sorted past patient list 82), and activity targets 84. The degree-of-similarity-sorted past patient list 82 includes identification information of the past patients selected in the degree-of-similarity-sorted past patient list 82, dates of admission, and motor ability values in respective items.

A case where the display screen shown in FIG. 14 is applied in the process in the step S108 will be described. In this case, the degree-of-similarity-sorted past patient list 82 shows a list of past patients corresponding to pieces of past patient information that are similar to the target patient information. When a user selects one of the past patients shown in the degree-of-similarity-sorted past patient list 82, the activity target 84 of that past patient is displayed. In the example shown in FIG. 14, since a past patient A is selected, the activity target 84 of the past patient A is displayed. The activity target 84 shows weekly activity targets for the corresponding past patient (the past patient A). When the past patient A was discharged from the hospital in a Wth week, the activity target in the Wth week is presented to the user as the long-term target candidate.

A case where the display screen shown in FIG. 14 is applied in the process in the step S118 will be described. In this case, the degree-of-similarity-sorted past patient list 82 shows a list of past patients corresponding to the long-term targets similar to the long-term target for the target patient. When a user selects one of the past patients displayed in the degree-of-similarity-sorted past patient list 82, the activity target 84 of that past patient is displayed. In the example shown in FIG. 14, since a past patient A is selected, the activity target 84 of the past patient A is displayed. When the past patient A was discharged from the hospital in a Wth week, the activity target at the middle point (e.g., in a wth week (w=W/2)) is presented to the user as the short-term target candidate.

As described above, the information processing apparatus 100 according to the first example embodiment determines, for each of a plurality of past patients, whether or not the long-term target of the past patient is similar to the long-term target for the target patient. Then, the information processing apparatus 100 according to the first example embodiment extracts short-term targets corresponding to the long-term targets that have been determined to be similar to the long-term target for the target patient, and performs a process for presenting the extracted short-term targets as short-term target candidates for the target patient.

Note that, as described above, the short-term targets in the past patient data can be those that have been created while taking the points of view of the three types of professions, e.g., a physical therapist, an occupational therapist, and a speech-language-hearing therapist into consideration. Therefore, the extracted short-term targets can be those that have been created while taking the points of view of the three types of professions into consideration. Accordingly, it is possible, by presenting the extracted short-term targets as the short-term target candidates for the target patient, to create proposed short-term targets in which the points of view of the three types of professions are taken into consideration. That is, when therapists in the above-described three types of professions meet and discuss short-term targets, they can select a short-term target from a limited number of short-term target candidates that are presented while taking the points of view of the three types of professions into consideration (or can determine a short-term target by using the limited number of short-term target candidates). Therefore, it is possible to determine a short-term target in a shorter time than in the case where short-term targets suitable for the target patient are examined one by one from among a myriad of short-term target candidates (which are not presented to the user). Therefore, the user (a therapist) can determine a short-term target in a short time.

Further, the information processing apparatus 100 according to the first example embodiment performs a process for presenting the extracted short-term targets as short-term target candidates for the target patient. In this way, the user (a therapist or the like) can create a short-term target easily and appropriately with reference to the presented short-term target candidates. Note that, instead of having the user determine a short-term target for the target patient, the information processing apparatus 100 may determine the short-term target corresponding to the long-term target of which the degree of similarity is the highest as the short-term target for the target patient. However, in the case where the information processing apparatus 100 determines the short-term target, there is a possibility that the individual circumstances of the target patient are not taken into consideration in the determination. Therefore, by presenting the extracted short-term targets as the short-term target candidates for the target patient, the user can create a short-term target in which the individual circumstances of the target patient are taken into consideration, so that a more appropriate short-term target can be created. Note that the short-term target can be a target provided as an intermediate milestone for the long-term target, and therefore a short-term target for a past patient of which the long-term target is similar to that for the target patient can also be an appropriate target for the target patient.

Further, the information processing apparatus 100 according to the first example embodiment determines whether or not the long-term target of each of a plurality of past patients is similar to the long-term target of the target patient. That is, the long-term target determination unit 140 according to the first example embodiment determines the degrees of similarity between the long-term targets of the past patients and the long-term target of the target patient. In many cases, when items in the long-term targets are similar to each other, short-term targets corresponding to the respective items are also similar to each other. Therefore, by comparing long-term targets with each other as in the case of the first example embodiment, it is possible to extract short-term targets suitable for the target patient in a more reliable manner.

### Second Example Embodiment

Next, a second example embodiment will be described with reference to the drawings. For clarifying the explanation, the following description and the drawings have been partially omitted and simplified as appropriate. Further, the same symbols are assigned to the same or corresponding components throughout the drawings and redundant descriptions thereof are omitted as appropriate. Note that since a system configuration according to the second example embodiment is substantially the same as that shown in FIG. 3, the description thereof is omitted. Further, since the configuration of an information processing apparatus 100 according to the second example embodiment is substantially the same as that shown in FIG. 4, the description thereof is omitted.

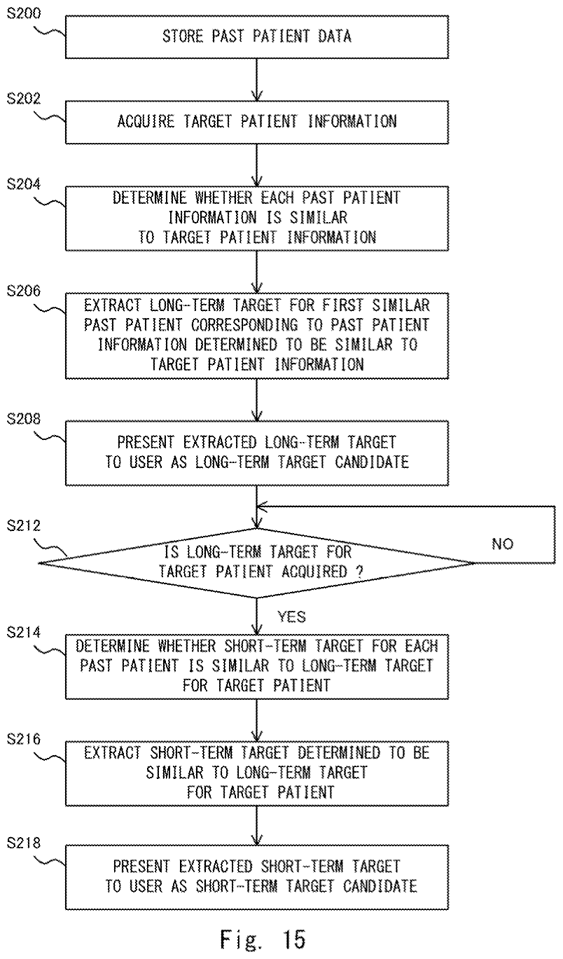

FIG. 15 is a flowchart showing a support method performed by the information processing apparatus 100 according to the second example embodiment. Note that processes in steps S200 to S212 are substantially the same as those in the steps S100 to S112, respectively, shown in FIG. 5, the descriptions thereof are omitted.

A long-term target determination unit 140 according to the second example embodiment determines whether or not a short-term target of each of a plurality of past patients is similar to the long-term target of the target patient (Step S214). That is, the long-term target determination unit 140 determines whether or not the activity target (the short-term target) of each of the plurality of past patients is similar to the long-term target of the activities (the rehabilitation) performed by the target patient. Specifically, the long-term target determination unit 140 determines whether or not a word(s) (a character string(s)) included in the activity target (the short-term target) of each of the plurality of past patients is similar to a word(s) (a character string(s)) included in the long-term target of the target patient.

The long-term target determination unit 140 calculates the degree of similarity between each of the short-term targets #1 to #N of the plurality of past patients and the long-term target 74I for the target patient. Then, the long-term target determination unit 140 may determine that short-term targets of which the degree of similarity is larger than or equal to a predetermined threshold are similar to the long-term target 74I for the target patient. Alternatively, the long-term target determination unit 140 may determine that short-term targets for past patients of which the degrees of similarity are within the top Nc3 ranks (Nc3 is a predetermined integer greater than or equal to one) are similar to the long-term target 74I for the target patient. Note that the method for calculating the degree of similarity may be substantially the same as those in the steps S104 and S114. However, the feature vectors used in the process in the step S214 are different from those used in the processes in the steps S104 and S114.

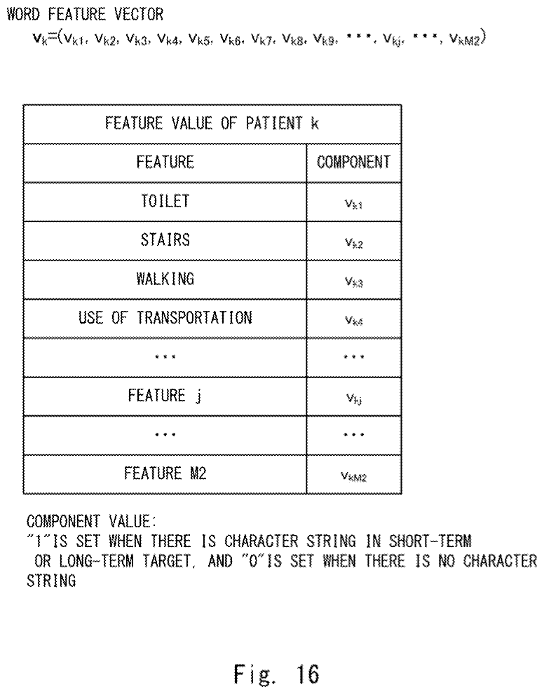

FIG. 16 shows an example of a word feature vector used in the calculation of the degree of similarity performed in the long-term target determination unit 140 according to the second example embodiment. The long-term target determination unit 140 according to the second example embodiment determines, by using this word feature vector, whether or not a word(s) (a character string(s)) included in the short-term target (the activity target) of each of the plurality of past patients is similar to a word(s) (a character string(s)) included in the long-term target 74I for the target patient. Note that, in the second example embodiment, the components of the word feature vector can be, for example, character strings indicating the items in the long-term target or the short-term target. That is, in the second example embodiment, when a component in the word feature vector corresponds to an evaluation item, the component of the word feature vector does not necessarily have to include the degree of independence corresponding to the evaluation item.

The word feature vector shown in FIG. 16 indicates the feature values in the long-term target for the target patient or in a short-term target for a past patient. Note that the word feature vector shown in FIG. 16 is merely an example, and various other word feature vectors can be used. Here, the total number of past patients is represented by N and the number of features is represented by M2 (M2 is an integer greater than or equal to one). Then, when a word feature vector of a patient k (a past patient k) is represented by vₖ, the M2 components of the feature vector are expressed by the below-shown Expression 9.

vₖ=(vₖ₁, vₖ₂, vₖ₃, vₖ₄, vₖ₅, vₖ₆, vₖ₇, vₖ₈, vₖ₉, . . . , vₖⱼ, . . . , v_{kM2}) (9)

In the example shown in FIG. 16, for example, a component vₖ₁ corresponds to a character string “Toilet” (an evaluation item) in the short-term target for a patient k. A component vₖ₂ corresponds to a character string “Stairs” (an evaluation item) in the short-term target for the patient k. A component vₖ₃ corresponds to a character string “Walking” (an evaluation item) in the short-term target for the patient k. A component vₖ₄ corresponds to a character string “Use of transportation” (an item) in the long-term target for the patient k. Note that the number of components M2 of the word feature vector, and what kind of feature each component value indicates may be determined by the user in advance.

Note that the component value of each component of the word feature vector may be assigned depending on whether or not the corresponding word is present in the short-term target. For example, when a character string “Toilet” is present in the short-term target for the patient k, the component value vₖ₁ is assigned “1” (vₖ₁=1), and when the character string “Toilet” is not present in the long-term target for the patient k, the component value vₖ₁ is assigned “0” (vₖ₁=0).

Further, a word feature vector vi of a target patient i is expressed by the below-shown Expression 10 as in the case of the Expression 9.

vᵢ=(vᵢ₁, vᵢ₂, vᵢ₃, vᵢ₄, vᵢ₅, vᵢ₆, vᵢ₇, vᵢ₈, vᵢ₉, . . . , vᵢⱼ, . . . , v_{iM2}) (10)

Note that each component vᵢⱼ corresponds to a character string in an item in the long-term target for the target patient. Further, the component values are substantially the same as those of the word feature vector of the patient k. Therefore, when a character string “Toilet” is present in the long-term target for the target patient, the component value vᵢ₁ is assigned “1” (vᵢ₁=1), and when the character string “Toilet” is not present in the long-term target for the target patient, the component value vi is assigned “0” (vᵢ₁=0)

The long-term target determination unit 140 calculates, for example, the degree of similarity Dw2ᵢₖ between the long-term target for the target patient i and the short-term target for the past patient k by the below-shown Expressions 11 and 12.

$$Dw {2}_{ik} = 1 / Sw {2}_{ik} \qquad ( 11 )$$

$$\begin{matrix}[ Expression 3 ] & \\ Sw {2}_{ik} = \sqrt{{\sum }_{j = 1}^{M 2} {( {v}_{ij} - {v}_{kj} )}^{2}} & ( 12 )\end{matrix}$$

The short-term target extraction unit 150 according to the second example embodiment extracts short-term targets that have been determined to be similar to the long-term target for the target patient in the process in the step S214 (Step S216). That is, the short-term target extraction unit 150 extracts short-term targets set for past patients corresponding to the activity targets (the short-term targets) that have been determined to be similar to the long-term target for the target patient. Then, similarly to the process in the step S118, the short-term target presentation unit 152 performs a process for presenting the short-term targets extracted in the process in the step S216 to the user as the short-term target candidates for the target patient (Step S218).

In many cases, items included in a long-term target for a given patient are similar to items included in a short-term target for that patient. Therefore, by configuring the system or the apparatus so that short-term targets for past patients that are similar to the long-term target for the target patient are extracted as in the case of the second example embodiment, it is possible to extract short-term targets without referring to the past patient data shown in FIG. 6. As a result, the process for extracting short-term targets is simplified.

Note that, in the above-described example, each component in the word feature vector is determined based on whether or not there is a word in a respective item. However, for items in each of which an evaluation item in the long-term target and the degree of independence thereof are specified, the corresponding components of the word feature vector may include the degree of independence in a manner similar to that in the first example embodiment as shown below.

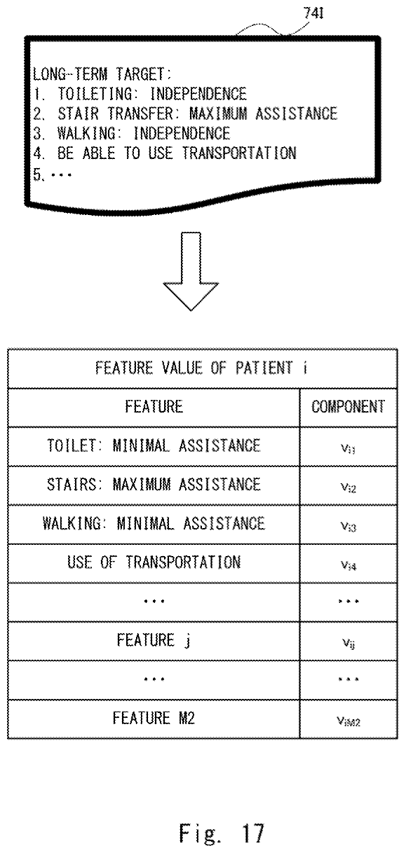

FIG. 17 shows another example of the word feature vector used in the calculation of the degree of similarity performed by the long-term target determination unit 140 according to the second example embodiment. In the example shown in FIG. 17, the long-term target determination unit 140 converts the degrees of independence corresponding to the respective evaluation items specified in the long-term target 74I for the target patient into such levels that, for example, the degrees of independence are reduced by half. That is, the long-term target determination unit 140 converts the degrees of independence (ability levels) corresponding to the respective items included in the long-term target 74I for the target patient into reduced degrees of independence (reduced ability levels) which are reduced according to a predetermined criterion. In the example shown in FIG. 17, the long-term target determination unit 140 converts the degree of independence “Independence” of each of the evaluation items “Toileting” and “Walking” included in the long-term target 74I into a reduced degree of independence “Minimal assistance”. Further, the long-term target determination unit 140 converts the degree of independence “Minimal assistance” of the evaluation item “Stair transfer” included in the long-term target 74I into a reduced degree of independence “Maximum assistance”. Note that when the degree of independence is expressed by a character string, the below-shown method may be used as the method for converting a degree of independence to a level thereof. The long-term target determination unit 140 determines whether there is a character string(s) indicating the degree of independence in each of the items in the long-term target 74I by using the above-described degree-of-independence dictionary, and extracts the character string(s) indicating the degree of independence. By using the above-described degree-of-independence dictionary, the long-term target determination unit 140 determines which level (numerical value) of the degree of independence the character string indicating the degree of independence in each of the items in the long-term target 74I represents. Then, the long-term target determination unit 140 determines the degree of independence that represents an intermediate level between the degree of independence of the character string and the lowest degree of independence as the reduced degree of independence. Note that the reduced degree of independence does not necessarily have to be exactly the middle “intermediate level”.

Then, as shown in FIG. 17, the long-term target determination unit 140 generates a word feature vector of a target patient i of which each of feature values is composed of a pair of a word in an evaluation item included in the long-term target 74I and a reduced degree of independence obtained by converting the degree of independence corresponding to the word. Then, the long-term target determination unit 140 determines, by using the word feature vector of the target patient i shown in FIG. 17 and the word feature vector of which each of the component (the feature values) is composed of a pair of an evaluation item included in the short-term target for the past patient and the degree of independence thereof, whether or not the short-term target for the past patient is similar to the long-term target for the target patient. That is, the long-term target determination unit 140 determines, for each of a plurality of past patients, whether or not the short-term target of the past patient is similar to the long-term target of the target patient by using each item and the corresponding reduced ability level, and each item and the corresponding ability level included in the short-term target of the past patient. By the above-described configuration, it is possible to extract short-term targets that are similar to the long-term target for the target patient in a more appropriate manner. That is, since the number of character strings indicating the degrees of independence increases as compared to the case where the feature values include only evaluation items, it is possible to prevent short-term targets of past patients of which the degrees of independence are not appropriate from being extracted.

### Third Example Embodiment

Next, a third example embodiment will be described with reference to the drawings. For clarifying the explanation, the following description and the drawings have been partially omitted and simplified as appropriate. Further, the same symbols are assigned to the same or corresponding components throughout the drawings and redundant descriptions thereof are omitted as appropriate. Note that since the system configuration according to the third example embodiment is substantially the same as that shown in FIG. 3, the description thereof is omitted. The third example embodiment differs from other example embodiments in that the patient information includes a patient individual keyword(s) corresponding to individual circumstances of a corresponding patient.

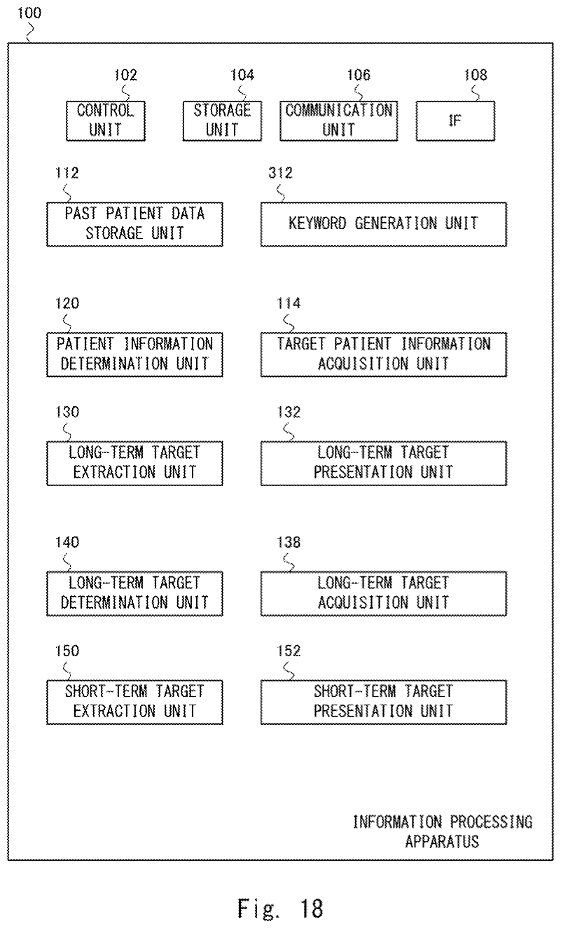

FIG. 18 shows a configuration of an information processing apparatus 100 according to the third example embodiment. The information processing apparatus 100 according to the third example embodiment includes a keyword generation unit 312. Other components/structures of the information processing apparatus 100 according to the third example embodiment are substantially the same as those shown in FIG. 4, and therefore descriptions thereof are omitted.

The keyword generation unit 312 has a function as keyword generation means. The keyword generation unit 312 generates a patient individual keyword by using patient individual information indicating a sentence expressing individual circumstances of a patient (past patients and the target patient). Note that the patient individual information is created for each patient. The patient individual information is text data (a sentence(s)) created by a therapist, the patient himself/herself, or a family member of the patient. The patient individual information indicates conditions and the like that the patient (or a family member or the like of the patient) wishes to have after the discharge from the hospital when the patient is admitted to a hospital.

The keyword generation unit 312 performs a morphological analysis for the patient individual information. The keyword generation unit 312 acquires a word(s) obtained by the morphological analysis as a patient individual keyword(s). Then, the keyword generation unit 312 adds the patient individual keyword(s) in the patient information (the past patient information and the target patient information). Note that the part of speech of the word acquired as the patient individual keyword may be restricted. For example, the keyword generation unit 312 may acquire, among the words obtained by the morphological analysis, words that are nouns as patient individual keywords. Alternatively, the keyword generation unit 312 may be configured so as not to acquire, among the words obtained by the morphological analysis, words that are particles, auxiliary verbs, or the like as patient individual keywords. Alternatively, a stop-word dictionary in which words (particles and the like) that are not appropriate as keywords are recorded may be stored in advance. In this case, the keyword generation unit 312 may be configured so as not to acquire, among the words obtained by the morphological analysis, words included in the stop-word dictionary as patient individual keywords.

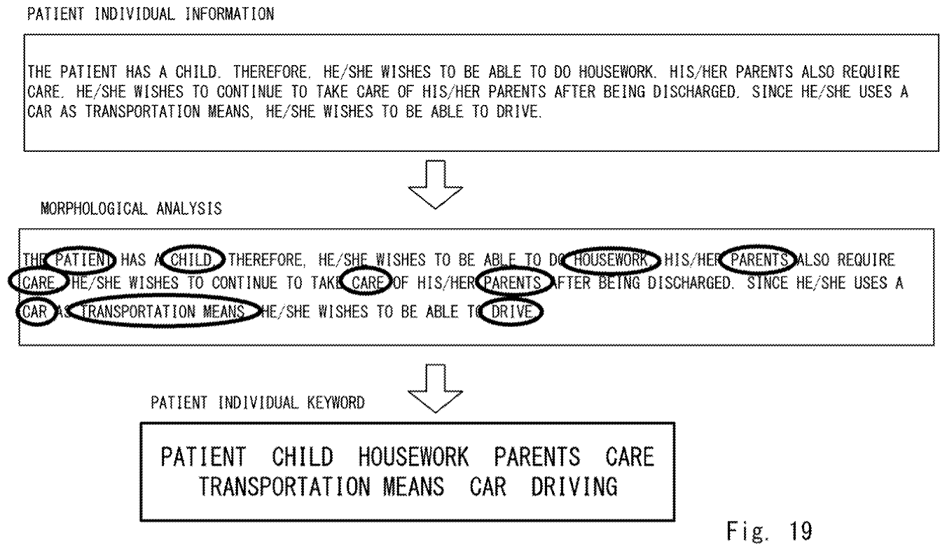

FIG. 19 is a diagram for explaining processing performed by the keyword generation unit 312 according to the third example embodiment. The patient individual information shown in FIG. 19 is created by a therapist. The keyword generation unit 312 performs a morphological analysis for the patient individual information shown in FIG. 19. As a result, the keyword generation unit 312 generates patient individual keywords “patient”, “child”, “housework”, “parents”, “care”, “transportation means”, “car”, and “driving” which correspond to the individual circumstances of the patient.

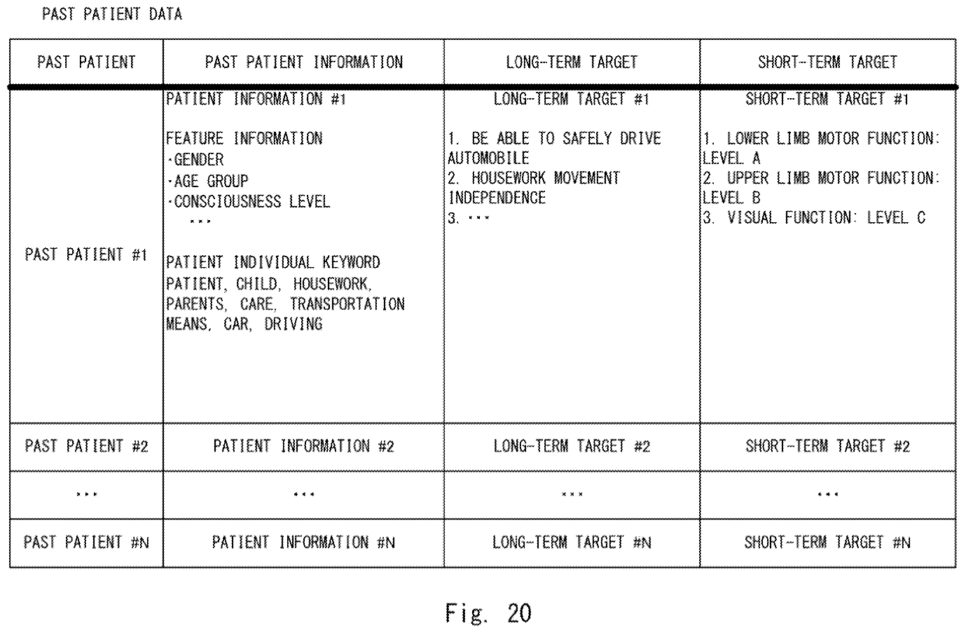

FIG. 20 is a table showing an example of past patient data according to the third example embodiment. Similarly to the example shown in FIG. 6, in the past patient data shown in FIG. 20, past patients, pieces of past patient information, long-term targets, and short-term targets are associated with each other. Note that the past patient information (the patient information) includes feature information and patient individual keywords generated by the keyword generation unit 312.

Further, in the third example embodiment, a long-term target associated with a past patient can include items in which the individual circumstances of the patient are taken into consideration as items different from the evaluation items in the FIM. In the third example embodiment, for example, a long-term target #1 of a past patient #1 includes “Be able to safely drive an automobile” and “Housework movement independence”. Further, in order to achieve the above-described items of the long-term target #1, a short-term target #1 can include items such as “Lower limb motor function: Level A”, “Upper limb motor function: Level B”, and “Visual function: Level C”. Note that these items in the long-term and short-term targets may also be included in the long-term and short-term targets according to the above-described example embodiments.

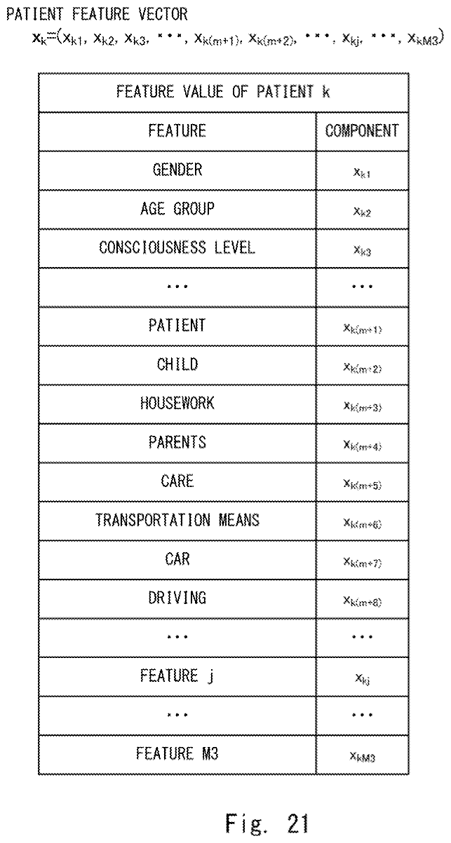

FIG. 21 shows an example of a patient feature vector used for the calculation of the degree of similarity performed in the patient information determination unit 120 according to the third example embodiment. The patient feature vector indicates feature values in the above-described patient information (the target patient information and the past patient information). Note that, in the third example embodiment, the feature values include feature information and patient individual keywords. Further, the patient feature vector shown in FIG. 21 is merely an example, and various other patient feature vectors can be used. FIG. 21 shows an example of a patient feature vector of a past patient, and the patient feature vector of the target patient is similar to this example as described later.

Here, the number of features is represented by M3 (M3 is an integer greater than or equal to one). When a patient feature vector of a patient k (a past patient k) is represented by xₖ, the M components of the feature vector are expressed by the below-shown Expression 13.

xₖ=(xₖ₁, xₖ₂, xₖ₃, . . . , xₖₘ, xₖ₍ₘ₊₁₎, xₖ₍ₘ₊₂₎, . . . , xₖⱼ, . . . , x_{kM3}) (13)

In the example shown in FIG. 21, components xₖ₍ₘ₊₁₎ to xₖ₍ₘ₊₈₎ correspond to patient individual keywords. For example, the component xₖ₍ₘ₊₁₎ corresponds to a keyword “patient”. Further, the component xₖ₍ₘ₊₃₎ corresponds to a keyword Further, the component xₖ₍ₘ₊₈₎ corresponds to a keyword “housework”. “driving”.

Note that the component value of each of the components related to the patient individual keywords can be assigned depending on whether or not the corresponding keyword is present in the patient information (the patient individual keyword). For example, when there is a keyword “housework” in a patient individual keyword of a patient k, the component value xₖ₍ₘ₊₃₎ is assigned “1” (xₖ₍ₘ₊₃₎=1), and when the keyword “housework” is not present in the patient individual keyword of the patient k, the component value xₖ₍ₘ₊₃₎ is assigned “0” (xₖ₍ₘ₊₃₎=0). This feature also applies to the other components. Note that since “child” and “boy/girl” are terms similar to each other, when a keyword “boy/girl” is present in the patient individual keyword of the patient k, the component value nₖ₍ₘ₊₂₎ is assigned “1” (xₖ₍ₘ₊₂₎=1).

Further, a patient feature vector xᵢ of a target patient i is expressed by the below-shown Expression 14 as in the case of the Expression 13.

xᵢ=(xᵢ₁, xᵢ₂, xᵢ₃, . . . , xᵢₘ, xᵢ₍ₘ₊₁₎, xᵢ₍ₘ₊₂₎, . . . xᵢⱼ, . . . , x_{iM3}) (14)

The patient information determination unit 120 calculates the degree of similarity Dpᵢₖ between the patient information of the target patient i (the target patient information) and the patient information of the past patient k (the past patient information) as shown in the above-shown Expressions 3 and 4. That is, the patient information determination unit 120 determines whether or not the past patient information is similar to the target patient information by using the patient individual keywords. The rest of the processes is substantially the same as those in the above-described example embodiments.

As described above, the information processing apparatus 100 according to the third example embodiment determines whether or not the past patient information is similar to the target patient information by using the patient individual keywords. In this way, the long-term target extraction unit 130 according to the third example embodiment can extract a long-term target in which the individual circumstances of the patient like those shown in FIG. 20 are taken into consideration. Further, since the long-term target in which the individual circumstances are taken into consideration can be extracted, it is possible to extract, from the short-term targets of the past patients, as a short-term target in which the individual circumstances are taken into consideration, a short-term target for achieving the long-term target for the target patient determined from the long-term targets in which the individual circumstances are taken into consideration. That is, the extracted short-term target can also be one in which the individual circumstances are taken into consideration. Further, the short-term target extraction unit 150 according to the third example embodiment can extract a short-term target including items for improving motor abilities and cognitive abilities in order to achieve the long-term target in which the individual circumstances of the patient like those shown in FIG. 20 are taken into consideration. Therefore, the information processing apparatus 100 according to the third example embodiment can present, to the user, long-term and short-term targets in which the individual circumstances of the target patient are taken into consideration. Therefore, the user can create long-term and short-term targets in which the individual circumstances of the target patient are taken into consideration.

For example, assume a case where an attribute of the target patient is “male in his 60s”, and “driving” is included in the patient individual keywords of the target patient. In this case, in the method according to the first example embodiment, since the individual circumstances related to “driving” are not taken into consideration, long-term targets of past patients having the attribute “male in his 60s” are extracted. As a result, short-term targets related to the past patients having the attribute “male in his 60s” can be extracted. In contrast, in the method according to the third example embodiment, individual circumstances related to “driving” are taken into consideration. Therefore, for example, when the individual keywords of a past patient include “driving”, the long-term target of that past patient can be extracted even when the past patient has an attribute that is different from the attribute of the target patient attribute, e.g., even when the past patient has an attribute “female in her 50s”. Then, as a result, a short-term target related to the past patient of which the patient individual keywords include “driving” can be extracted irrespective of his/her other attributes. As described above, in the method according to the third example embodiment, long-term and short-term targets related to the individual circumstances of the target patient can be extracted. In this way, the user can easily create long-term and short-term targets in which the individual circumstances of the target patient are taken into consideration.

### Modified Example

Note that the present invention is not limited to the above-described example embodiments, and they can be modified as appropriate without departing from the scope and spirit of the invention. For example, the above-described plurality of example embodiments can be applied to one another. For example, the configuration according to the third example embodiment may be combined with that according to the second example embodiment. The same is true for the other example embodiments.

Further, in each of the above-described flowcharts, the order of processes (steps) can be changed as appropriate. Further, at least one of a plurality of processes (steps) may be omitted (or skipped). For example, the steps S102 to S108 in FIG. 5 are not indispensable. The same is true for FIG. 15. That is, a user may create a long-term target for a target patient without using the information processing apparatus 100 according to the example embodiment.

Further, although the information processing apparatus 100 according to the above-described example embodiment supports the creation of plans (long-term and short-term targets) for rehabilitation, the activities to which the example embodiment is applied are not limited to the rehabilitation. The example embodiment can be applied to any activities aimed at improving abilities. For example, the example embodiment can be applied to habilitation. Further, for example, the example embodiment can also be applied to activities for improving sport abilities.

In the above-described examples, the program may be stored in various types of non-transitory computer readable media and thereby supplied to computers. The non-transitory computer readable media includes various types of tangible storage media. Examples of the non-transitory computer readable media include a magnetic recording medium (such as a flexible disk, a magnetic tape, and a hard disk drive), a magneto-optic recording medium (such as a magneto-optic disk), a CD-ROM (Read Only Memory), CD-R, CD-R/W, and a semiconductor memory (such as a mask ROM, a PROM (Programmable ROM), an EPROM (Erasable PROM), a flash ROM, and a RAM (Random Access Memory)). Further, the programs may be supplied to computers by using various types of transitory computer readable media. Examples of the transitory computer readable media include an electrical signal, an optical signal, and an electromagnetic wave. The transitory computer readable media can be used to supply programs to a computer through a wired communication line (e.g., electric wires and optical fibers) or a wireless communication line.

Further, the whole or part of the example embodiments disclosed above can be described as, but not limited to, the following supplementary notes.

(Supplementary Note 1)

An information processing apparatus comprising:

storage means for storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients:

long-term target determination means for determining whether or not the activity target of each of the plurality of past patient is similar to a long-term target of the activity of a target patient:

short-term target extraction means for extracting the short-term target set for the past patient corresponding to the activity target determined to be similar to the long-term target for the target patient: and

short-term target presentation means for performing a process for presenting information about the extracted short-term target.

(Supplementary Note 2)

The information processing apparatus described in Supplementary note 1, wherein

the storage means stores the past patient data in which the plurality of past patients are associated with activity targets each of which includes at least the long-term and short-term targets for the activity performed by a respective one of the plurality of past patients,

the long-term target determination means determines, for each of the plurality of past patients, whether or not the long-term target of the past patient is similar to the long-term target for the target patient, and

the short-term target extraction means extracts the short-term target set for the past patient corresponding to the long-term target determined to be similar to the long-term target for the target patient.

(Supplementary Note 3)

The information processing apparatus described in Supplementary note 1, wherein

the long-term target determination means determines, for each of the plurality of past patients, whether or not the short-term target of the past patient is similar to the long-term target for the target patient, and

the short-term target extraction means extracts the short-term target determined to be similar to the long-term target for the target patient.

(Supplementary Note 4)

The information processing apparatus described in Supplementary note 3, wherein the long-term target determination means converts ability levels each of which corresponds to a respective one of items included in the long-term target for the target patient into reduced ability levels reduced according to a predetermined criterion, and determines, for each of the plurality of past patients, whether or not the short-term target of the past patient is similar to the long-term target for the target patient by using each of the items and the corresponding reduced ability level, and each of the items included in the short-term target of the past patient and the corresponding ability level.

(Supplementary Note 5)

The information processing apparatus described in any one of Supplementary notes 1 to 4, wherein the short-term target presentation means performs a process for presenting the extracted short-term target to the user as a candidate for the short-term target for the target patient.

(Supplementary Note 6)

The information processing apparatus described in any one of Supplementary notes 1 to 5, wherein the long-term target determination means determines, for each of the plurality of past patients, whether or not a character string included in the activity target of the past patient is similar to a character string included in the long-term target for the target patient.

(Supplementary Note 7)

The information processing apparatus described in any one of Supplementary notes 1 to 6, wherein

the storage means stores the past patient data in which the plurality of past patients, pieces of past patient information each of which indicates at least a feature of a respective one of the plurality of past patients, and activity targets each of which includes at least the long-term and short-term targets for the activity performed by a respective one of the plurality of past patients are associated with each other, and

the information processing apparatus further comprises:

patient information determination means for determining, for each of the plurality of past patients, whether or not the past patient information of the past patient is similar to target patient information indicating at least the feature of the target patient:

long-term target extraction means for extracting the long-term target set for the past patient corresponding to the past patient information determined to be similar to the target patient information; and

long-term target presentation means for performing a process for presenting information about the extracted long-term target.

(Supplementary Note 8)

The information processing apparatus described in Supplementary note 7, wherein

the past patient information includes a patient individual keyword indicating an individual circumstance of a corresponding past patient, and the target patient information includes a patient individual keyword indicating an individual circumstance of a corresponding target patient, and

the patient information determination means determines whether or not the past patient information is similar to the target patient information by using the individual patient keywords.

(Supplementary Note 9)

The information processing apparatus described in Supplementary note 8, further comprising keyword generation means for generating the patient individual keyword by performing a morphological analysis for patient individual information indicating a sentence expressing an individual circumstance of a patient.

(Supplementary Note 10)

The information processing apparatus described in any one of Supplementary notes 7 to 9, wherein the long-term target presentation means performs a process for presenting the extracted long-term target to the user as a candidate for the long-term target for the target patient.

(Supplementary Note 11)

A support method comprising: storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients:

determining whether or not the activity target of each of the plurality of past patient is similar to a long-term target of the activity of a target patient:

extracting the short-term target set for the past patient corresponding to the activity target determined to be similar to the long-term target for the target patient: and

performing a process for presenting information about the extracted short-term target.

(Supplementary Note 12)

The support method described in Supplementary note 11, further comprising:

storing the past patient data in which the plurality of past patients are associated with activity targets each of which includes at least the long-term and short-term targets for the activity performed by a respective one of the plurality of past patients:

determining, for each of the plurality of past patients, whether or not the long-term target of the past patient is similar to the long-term target for the target patient: and

extracting the short-term target set for the past patient corresponding to the long-term target determined to be similar to the long-term target for the target patient.

(Supplementary Note 13)

The support method described in Supplementary note 11, further comprising:

determining, for each of the plurality of past patients, whether or not the short-term target of the past patient is similar to the long-term target for the target patient: and

extracting the short-term target determined to be similar to the long-term target for the target patient.

(Supplementary Note 14)

The support method described in Supplementary note 13, further comprising converting ability levels each of which corresponds to a respective one of items included in the long-term target for the target patient into reduced ability levels reduced according to a predetermined criterion, and determining, for each of the plurality of past patients, whether or not the short-term target of the past patient is similar to the long-term target for the target patient by using each of the items and the corresponding reduced ability level, and each of the items included in the short-term target of the past patient and the corresponding ability level.

(Supplementary Note 15)

The support method described in any one of Supplementary notes 11 to 14, further comprising performing a process for presenting the extracted short-term target to the user as a candidate for the short-term target for the target patient.

(Supplementary Note 16)

The support method described in any one of Supplementary notes 11 to 15, further comprising determining, for each of the plurality of past patients, whether or not a character string included in the activity target of the past patient is similar to a character string included in the long-term target for the target patient.

(Supplementary Note 17)

The support method described in any one of Supplementary notes 11 to 16, further comprising:

storing the past patient data in which the plurality of past patients, pieces of past patient information each of which indicates at least a feature of a respective one of the plurality of past patients, and activity targets each of which includes at least the long-term and short-term targets for the activity performed by a respective one of the plurality of past patients are associated with each other:

determining, for each of the plurality of past patients, whether or not the past patient information of the past patient is similar to target patient information indicating at least the feature of the target patient:

extracting the long-term target set for the past patient corresponding to the past patient information determined to be similar to the target patient information: and

performing a process for presenting information about the extracted long-term target.

(Supplementary Note 18)

The support method described in Supplementary note 17, wherein

the past patient information includes a patient individual keyword indicating an individual circumstance of a corresponding past patient, and the target patient information includes a patient individual keyword indicating an individual circumstance of a corresponding target patient, and further comprising:

determining whether or not the past patient information is similar to the target patient information by using the individual patient keywords.

(Supplementary Note 19)

The support method described in Supplementary note 18, further comprising generating the patient individual keyword by performing a morphological analysis for patient individual information indicating a sentence expressing an individual circumstance of a patient.

(Supplementary Note 20)

The support method described in any one of Supplementary notes 17 to 19, further comprising performing a process for presenting the extracted long-term target to the user as a candidate for the long-term target for the target patient.

(Supplementary Note 21)

A non-transitory computer readable medium storing a program for causing a computer to perform:

a function of storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients:

a function of determining whether or not the activity target of each of the plurality of past patient is similar to a long-term target of the activity of a target patient:

a function of extracting the short-term target set for the past patient corresponding to the activity target determined to be similar to the long-term target for the target patient: and

a function of performing a process for presenting information about the extracted short-term target.

Although the present invention is described above with reference to example embodiments, the present invention is not limited to the above-described example embodiments. Various modifications that can be understood by those skilled in the art can be made to the configuration and details of the present invention within the scope of the invention.

This application is based upon and claims the benefit of priority from Japanese patent application No. 2019-197791, filed on Oct. 30, 2019, the disclosure of which is incorporated herein in its entirety by reference.

### Reference Signs List

1 INFORMATION PROCESSING APPARATUS
2 STORAGE UNIT
12 LONG-TERM TARGET DETERMINATION UNIT
14 SHORT-TERM TARGET EXTRACTION UNIT
16 SHORT-TERM TARGET PRESENTATION UNIT
50 SUPPORT SYSTEM
60 USER TERMINAL
72 PATIENT INFORMATION
74 LONG-TERM TARGET
76 SHORT-TERM TARGET
80 DISPLAY SCREEN
82 DEGREE-OF-SIMILARITY-SORTED PAST PATIENT LIST
84 ACTIVITY TARGET
100 INFORMATION PROCESSING APPARATUS
112 PAST PATIENT DATA STORAGE UNIT
114 TARGET PATIENT INFORMATION ACQUISITION UNIT
120 PATIENT INFORMATION DETERMINATION UNIT
130 LONG-TERM TARGET EXTRACTION UNIT
132 LONG-TERM TARGET PRESENTATION UNIT
138 LONG-TERM TARGET ACQUISITION UNIT
140 LONG-TERM TARGET DETERMINATION UNIT
150 SHORT-TERM TARGET EXTRACTION UNIT
152 SHORT-TERM TARGET PRESENTATION UNIT
312 KEYWORD GENERATION UNIT

What is claimed is:

1. An information processing apparatus comprising:
hardware, including a processor and memory;
storage unit implemented at least by the hardware and configured to store past patient data in which a plurality of past patients are associated with activity targets, the plurality of past patients being patients who performed an activity aimed at improving their abilities in a past, and each of the activity targets including at least a short-term target for the activity of a respective one of the plurality of past patients;
long-term target determination unit implemented at least by the hardware and configured to determine whether or not the activity target of each of the plurality of past patient is similar to a long-term target of the activity of a target patient;
short-term target extraction unit implemented at least by the hardware and configured to extract the short-term target set for the past patient corresponding to the activity target determined to be similar to the long-term target for the target patient; and
short-term target presentation unit implemented at least by the hardware and configured to perform a process for presenting information about the extracted short-term target.

2. The information processing apparatus according to claim 1, wherein
the storage unit stores the past patient data in which the plurality of past patients are associated with activity targets each of which includes at least the long-term and short-term targets for the activity performed by a respective one of the plurality of past patients,
the long-term target determination unit determines, for each of the plurality of past patients, whether or not the long-term target of the past patient is similar to the long-term target for the target patient, and
the short-term target extraction unit extracts the short-term target set for the past patient corresponding to the long-term target determined to be similar to the long-term target for the target patient.

3. The information processing apparatus according to claim 1, wherein
the long-term target determination unit determines, for each of the plurality of past patients, whether or not the short-term target of the past patient is similar to the long-term target for the target patient, and
the short-term target extraction unit extracts the short-term target determined to be similar to the long-term target for the target patient.

4. The information processing apparatus according to claim 3, wherein the long-term target determination unit converts ability levels each of which corresponds to a respective one of items included in the long-term target for the target patient into reduced ability levels reduced according to a predetermined criterion, and determines, for each of the plurality of past patients, whether or not the short-term target of the past patient is similar to the long-term target for the target patient by using each of the items and the corresponding reduced ability level, and each of the items included in the short-term target of the past patient and the corresponding ability level.

5. The information processing apparatus according to claim 1, wherein the short-term target presentation unit performs a process for presenting the extracted short-term target to the user as a candidate for the short-term target for the target patient.

6. The information processing apparatus according to claim 1, wherein the long-term target determination unit determines, for each of the plurality of past patients, whether or not a character string included in the activity target of the past patient is similar to a character string included in the long-term target for the target patient.

7. The information processing apparatus according to claim 1, wherein
the storage unit stores the past patient data in which the plurality of past patients, pieces of past patient information each of which indicates at least a feature of a respective one of the plurality of past patients, and activity targets each of which includes at least the long-term and short-term targets for the activity performed by a respective one of the plurality of past patients are associated with each other, and the information processing apparatus further comprises:
patient information determination unit implemented at least by the hardware and configured to determine, for each of the plurality of past patients, whether or not the past patient information of the past patient is similar to target patient information indicating at least the feature of the target patient;
long-term target extraction unit implemented at least by the hardware and configured to extract the long-term target set for the past patient corresponding to the past patient information determined to be similar to the target patient information; and
long-term target presentation unit implemented at least by the hardware and configured to perform a process for presenting information about the extracted long-term target.

8. The information processing apparatus according to claim 7, wherein
the past patient information includes a patient individual keyword indicating an individual circumstance of a corresponding past patient, and the target patient information includes a patient individual keyword indicating an individual circumstance of a corresponding target patient, and
the patient information determination unit determines whether or not the past patient information is similar to the target patient information by using the individual patient keywords.

9. The information processing apparatus according to claim 8, further comprising keyword generation unit implemented at least by the hardware and configured to generate the patient individual keyword by performing a morphological analysis for patient individual information indicating a sentence expressing an individual circumstance of a patient.

10. The information processing apparatus according to claim 7, wherein the long-term target presentation unit performs a process for presenting the extracted long-term target to the user as a candidate for the long-term target for the target patient.

11. A support method comprising:
storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients;
determining whether or not the activity target of each of the plurality of past patient is similar to a long-term target of the activity of a target patient;
extracting the short-term target set for the past patient corresponding to the activity target determined to be similar to the long-term target for the target patient; and
performing a process for presenting information about the extracted short-term target.

12. The support method according to claim 11, further comprising:
storing the past patient data in which the plurality of past patients are associated with activity targets each of which includes at least the long-term and short-term targets for the activity performed by a respective one of the plurality of past patients;
determining, for each of the plurality of past patients, whether or not the long-term target of the past patient is similar to the long-term target for the target patient; and
extracting the short-term target set for the past patient corresponding to the long-term target determined to be similar to the long-term target for the target patient.

13. The support method according to claim 11, further comprising:
determining, for each of the plurality of past patients, whether or not the short-term target of the past patient is similar to the long-term target for the target patient; and
extracting the short-term target determined to be similar to the long-term target for the target patient.

14. The support method according to claim 13, further comprising converting ability levels each of which corresponds to a respective one of items included in the long-term target for the target patient into reduced ability levels reduced according to a predetermined criterion, and determining, for each of the plurality of past patients, whether or not the short-term target of the past patient is similar to the long-term target for the target patient by using each of the items and the corresponding reduced ability level, and each of the items included in the short-term target of the past patient and the corresponding ability level.

15. The support method according to claim 11, further comprising performing a process for presenting the extracted short-term target to the user as a candidate for the short-term target for the target patient.

16. The support method according to claim 11, further comprising determining, for each of the plurality of past patients, whether or not a character string included in the activity target of the past patient is similar to a character string included in the long-term target for the target patient.

17. The support method according to claim 11, further comprising:
storing the past patient data in which the plurality of past patients, pieces of past patient information each of which indicates at least a feature of a respective one of the plurality of past patients, and activity targets each of which includes at least the long-term and short-term targets for the activity performed by a respective one of the plurality of past patients are associated with each other;
determining, for each of the plurality of past patients, whether or not the past patient information of the past patient is similar to target patient information indicating at least the feature of the target patient;
extracting the long-term target set for the past patient corresponding to the past patient information determined to be similar to the target patient information; and
performing a process for presenting information about the extracted long-term target.

18. The support method according to claim 17, wherein
the past patient information includes a patient individual keyword indicating an individual circumstance of a corresponding past patient, and the target patient information includes a patient individual keyword indicating an individual circumstance of a corresponding target patient, and further comprising:
determining whether or not the past patient information is similar to the target patient information by using the individual patient keywords.

19. (Canceled)

20. The support method according to claim 17, further comprising performing a process for presenting the extracted long-term target to the user as a candidate for the long-term target for the target patient.

21. A non-transitory computer readable medium storing a program for causing a computer to perform:
a function of storing past patient data in which a plurality of past patients are associated with activity targets, the plurality of past patients being patients who performed an activity aimed at improving their abilities in a past, and each of the activity targets including at least a short-term target for the activity of a respective one of the plurality of past patients;
a function of determining whether or not the activity target of each of the plurality of past patient is similar to a long-term target of the activity of a target patient;
a function of extracting the short-term target set for the past patient corresponding to the activity target determined to be similar to the long-term target for the target patient; and
a function of performing a process for presenting information about the extracted short-term target.
